(19) United States
(12) Patent Application Publication
(10) Pub. No.: US 2003/0130272 A1
(43) Pub. Date: Jul. 10, 2003

(54) Anthracene derivatives as anti-cancer agents

(76) Inventors: David John Mincher, Edinburgh (GB); Agnes Turnbull, Edinburgh (GB); Graeme Gillies Kay, Edinburgh (GB)

Correspondence Address: Nixon & Vanderhye, 8th Floor, 1100 North Glebe Road, Arlington, VA, 22201-4714, US

(21) Appl. No.: 10/149,580

(22) PCT Filed: Dec. 15, 2000
(86) PCT No.: PCT/GB00/04829

(30) Foreign Application Priority Data
Dec. 16, 1999 (GB) 99298010

(51) Int. Cl. A61K 31/54; A61K 31/537; A61K 31/495; A61K 31/445; A61K 31/40; A61K 31/137

(52) U.S. Cl. 514/227.5; 514/649; 514/231.2; 514/252.12; 514/325; 514/408

(57) ABSTRACT

Use of compound of Formula (I): at least one of R¹, R², R⁵ and R⁶ is a group —AB and the others are independently selected from hydrogen, hydroxy, alkoxy or acyloxy, a group —AB a group -amino-(R⁷)ₙX—Y wherein R⁷ is a divalent organic radical and n is 0 or 1; R³ and R⁴are independently oxo, hydroxy or hydrogen; the or each A is independently a spacer group of formula -amino-(R⁷)ₙ—X— which is bonded to the anthracene ring via the amino group nitrogen and to B via —X—, X is independently selected from O, NH and C(O); B is an amino acid residue or a peptide group or isostere thereof and Y is hydrogen or a capping group, or a physiologically acceptable derivative of such compound for the manufacture of a medicament for the treatment of cancers or microbial infections having cells exhibiting topoisomerase I activity characterised in that the group -amino-(R⁷)ₙ—X— incorporates an optionally substituted heterocyclic ring directly attached to the anthroquinone ring through an amino nitrogen in the heterocycclic ring, or an optionally substituted heterocyclic or carbocyclic ring that is spaced from the anthraquinone ring by no more than an amino nitrogen and up to four carbon atoms.

The present invention relates to compounds which are based on an anthraquinone nucleus for use in medicine, particularly as anti-cancer agents which exert their effects, at least in part, through their interaction with the activity of topoisomerases.

The inhibition of DNA topoisomerases, particularly topoisomerase II (topo II) is now considered to be an important component in the mechanism of action of a large number of the most clinically active anticancer drugs presently available, including doxorubicin, mitoxantrone, VP16, camptothecin, topotecan, M-AMSA, VM26 and the ellipiticines. These drugs are believed to inhibit topo II by stabilising a protein/drug/nucleic acid ternary complex termed the cleavable complex.

However, whilst targeting topoisomerases, the aforesaid prior art drugs also exhibit a number of other mechanisms of action, such as generation of free radicals and formation of DNA covalent adducts which contribute to their overall toxicity and poor therapeutic index. Additionally, the failure of these agents to produce long term cures in the major malignancies is probably exacerbated by the presence of de novo resistance and the development of acquired drug resistance.

U.S. Pat. No. 5,733,880 and its corresponding EP 0721447 disclose compounds of general formula wherein R¹ and R² are independently hydrogen or hydroxyl, R³ and R⁴ are

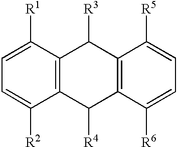

independently oxo or hydrogen, one of R⁵ and R⁶ is A—B and the other is hydrogen, hydroxyl or a group A, wherein the or each A is independently a spacer group providing NH or CO in the bond with B, if present, at least one group A does not provide the residue of an α-amino acid adjacent the anthraquinone nucleus and the A of any A—B moiety is joined to the anthraquinone nucleus via an —NH— bond, and the or each B is a peptide group or a physiologically acceptable derivative thereof. These compounds are described as being particularly useful antitumour compounds acting as topoisomerase inhibitors and also to be useful as dyes.

Copending PCT/GB99/01901 discloses novel anthraquinones of the same type as U.S. Pat. No. 5,733,880, but where the bond between the groups A and B is provided by an oxygen atom on group A and the C-terminal carbonyl on group B, thus providing an ester linkage between the two. These compounds are disclosed as topoisomerase inhibitors with advantages of reduced free radical generation, reduced DNA or RNA binding and an activity profile more directed towards topoisomerases I and IIβ than topoisomerase IIα as compared to existing compounds. At least some of these compounds are less active, in vitro at least, than the corresponding anide linked compounds of U.S. Pat. No. 5,733,880.

It is an object of the present invention to provide novel use of known and novel compounds to more specifically target topoisomerase I (topo I) than topoisomerase IIα or IIβ such that they will have relatively greater activity toward cancers and micro-organisms which have significant topo I expression, particularly toward those having greater topo I activity than that of either of the topo II's. It will be realised that topo I mediated activity will be more likely to be effective than topo II mediated activity in cell lines that are resistant to topo II agents.

Topo I is an anti-cancer target of growing importance as it is commonly over-expressed in tumour tissue compared to normal tissue and, significantly, this is not proliferation dependent. Despite a number of new anti-topo I agents being identified, clinical applications are at present confined to derivatives of camptothecin. All these compounds, and camptothecin, have two major inherent limitations: (i) they are in equilibrium with their inactive carboxylate form at physiological pH and (ii) their topo I cleavage complexes reverse within minutes of drug removal thus imposing long infusions upon those undergoing therapy.

The present inventors have now determined that the introduction of appropriately positioned, conformationally restricted groups into the spacer group of spacer-linked anthraquinone-amino acid or peptide conjugates, allows control of the affinity and mode of binding to DNA. This affinity and mode of binding to DNA is readily determined by fluorescence quenching experiments in which the Q₅₀ values for displacement of ethidium bromide, a DNA intercalator, and for Hoechst 33258 dye, a DNA minor-groove-binder, from which their fluorescent DNA-bound complexes are measured and expressed in μM units of drug concentration (see PCT/GB99/01901 incorporated herein by reference).

The present inventors have previously demonstrated that prior art anthraquinone-amino acid or -peptide conjugates bind to DNA by a mixed modal binding mode, i.e. part-intercalative, part-groove binding mode, in common with a number of anthracyclines including adriamycin, daunomycin, the naturally occurring echinomycin and the synthetic anti-cancer drug mitoxantrone.

In general the anthraquinone-amino acid conjugates of U.S. Pat. No. 5,733,880 and PCT/GB99/01901 bind less strongly to DNA than mitoxantrone which has Q_{H50} 1.0 μM and Q_{E50} 0.35 μM for groove-binding and intercalation respectively. The in vitro and in vivo active conjugate NU:UB 31 of U.S. Pat. No. 5,733,880, containing the terminally-cyclic proline motif has corresponding Q values of 1.3 and 1.4 μM respectively.

The present inventors have now provided compounds which bind to DNA principally by groove-binding and preferred compounds stimulate topo I-mediated cleavage of DNA without affecting the ability of the enzyme to relax DNA. The groove-binding capacity and topo I activity can be related directly to the presence of the rigid conformationally restricted spacer moieties which control the extent of groove binding and preferably improve contact with the enzyme. Conversely, increased intercalative binding correlates with topo II activity.

Groove binding agents are known to stimulate topo 1-mediated cleavage of DNA [H. Wang, R. Gupta and J. W. Lown, (1994), Synthesis, DNA binding, sequence preference and biological evaluation of minor groove-selective N1-alkoxyalkyl-bis-benzimidazoles, Anti-Cancer Drug Design, 9, 153-180.]

Preferred compounds of the present invention prevent DNA strand re-ligation after drug induced DNA damage and, as with camptothecin, catalytic relaxation activity of the topo I remains unaffacted. Unlike some dual topo I/topo II inhibitors of the anthraquinone type, these compounds do not therefor antagonise their own cleavage reaction at high concentrations.

According to a first aspect of the invention there is provided the use of compound having the Formula I:

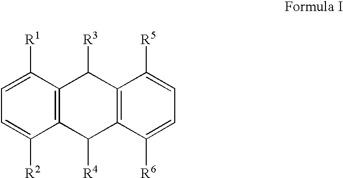

wherein

at least one of R¹, R², R⁵ and R⁶ is a group —AB and the others are independently selected from hydrogen, hydroxy, alkoxy, acyloxy, a group —AB and a group -amino-(R⁷)ₙX—Y wherein R⁷ is a divalent organic radical and n is 0 or 1;

R³ and R⁴ are independently oxo, hydroxy or hydrogen;

the or each A is independently a spacer group of formula -amino-(R⁷)ₙ—X— which is bonded to the anthracene ring via the amino group nitrogen and to B via —X—

X is independently selected from O, NH and C(O);

B is an independently selected amino acid residue or a peptide group or isostere thereof; and

Y is hydrogen or a capping group,

or a physiologically acceptable derivative of such compound for the manufacture of a medicament for the treatment of cancers or microbial infections having cells exhibiting topoisomerase I activity.

characterised in that the group -amino-(R⁷)ₙ—X— incorporates an optionally substituted heterocyclic ring directly attached to the anthroquinone ring through an amino nitrogen in the heterocyclic ring, or R⁷ when present includes an optionally substituted heterocyclic or carbocyclic ring that is spaced from the anthraquinone ring by no more than an amino nitrogen and up to four carbon atoms.

Particularly when R⁷ includes the ring it is spaced from the amino group attached to the anthraquinone ring by no more than two carbons, preferably no more than one carbon and more preferably is directly attached to the amino group nitrogen.

Particularly the medicarnent is for treatment of cancers or microbial infections wherein the cancer or microbe topoisomerase I activity is greater than that of healthy human cells, particularly non-dividing human cells eg. colon, lung, skin or other cells subject to occurrence of neoplasms, eg. healthy cells of the patient requiring treatment.

Preferably R¹, R², R⁵ and R⁶, when not AB or -amino-(R⁷)ₙ—X—Y are independently selected from hydrogen and hydroxy, but when they are alkoxy or acyloxy, these are preferably selected from C₁₋₆ alkoxy or acyloxy, such as methoxy and ethoxy, or acetoxy and propionyloxy.

Clearly, when R³ or R⁴ are oxo, the single line to the ring represents a double bond.

Preferred compounds are those of Formula II

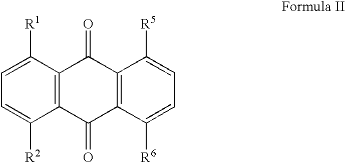

Preferably only one of R¹, R², R⁵ and R⁶ is a group —A—B and the others are independently selected from hydrogen, hydroxy, alkoxy, acyloxy, more preferably hydrogen or hydroxy.

The term amino, as used with respect to -amino-R⁷—X—, may be a group —NH—, —NR¹⁰— or —N<R¹¹—. R¹⁰ is selected from alkyl, alkenyl, aralkyl or aryl, most preferably being alkyl. All R¹⁰ groups alkyl, alkenyl, aralkyl or aryl preferably contain only one or two C₁₋₆ alkyl groups and/or a single phenyl ring as appropriate.

When the, or one of the, optionally substituted heterocyclic rings is present in the -amino- portion of -amino-(R⁷)ₙ—X—, this is of formula is —N<R¹¹—, where R¹¹ consists of a moiety with which the —N< makes up a heterocylic ring system, preferably a single heterocyclic ring, containing the nitrogen atom of the aforesaid —N< moiety and up to 6, but preferably only 3, 4 or 5 other members selected from nitrogen, oxygen, sulphur and carbon. Most preferably such amino group is a ring selected from NC₄, NC₅, N₂C₃ and N₂C₄ rings, ie. pyrrole, 2H-pyrrole, pyrrolidine, pyrroline, imidazole, imidazidine, imidazoline, pyrazole, pyrazolidine, pyrazoline, pyridine, pyrazine, piperidine, and piperazine. —R⁷— may be bonded to any of the atoms of the moiety completing the ring.

Preferably the or each A is independently a spacer group having the formula —NH—R⁷—NH— or —N<R¹¹—, where R¹¹ includes a further amino nitrogen, which group is bonded to the anthracene nucleus via the leading —NH— or —N< moiety and to B via the trailing —NH— moiety or further amino nitrogen in each case. Preferably one A only is linked to B. Preferably one of R⁵ and R⁶ is hydrogen or hydroxy.

R⁷ may be any divalent group that spaces the moiety —X— from the amino group on the anthracine ring system by a contiguous chain of 1 to 20 atoms, more preferably 1 to 12 atoms, and most preferably 2 to 6 atoms especially 3, 4 or 5 atoms.

Where the amino does not incorporate a heterocyclic ring, the group R⁷ consists of or includes one or more carbocyclic or heterocyclic rings which may spaced or flanked, on one or both sides, by one or more straight or branched alkylene chains. These rings may be saturated or unsaturated. The alkylene chain may alternatively or additionally be interrupted by an olefinic bond or by one or more hetero atoms such as in —O—, —S—, —S—S—, —NH—, —N(C₁₋₆alkyl)-. It will be realised that the —NH— may require protection eg. by Boc, during syhthesis of the compounds for use in the invention, depending on synthetic route.

R⁷ may be branched, eg, by way of substituents on the alkylene chain such as halo, hydroxy, C₁₋₆ alkyl, C₁₋₆ hydroxyalkyl, C₁₋₆ alkoxy, C₇₋₁₂aralkyl, eg benzyl. Further examples include groups including chiral centre carbons in the chain, eg. where the alkylene chain is substituted with alkyl such as in —CH(C₂H₅)—(CH₂)₂—X—, and gem-dialkyl groups centred on chain carbon atoms, such as in —C(CH₃)₂—(CH₂)₂—X—

The most preferred groups -amino-(R⁷)ₙX— fall into two groups:

(i) those where -amino- comprises a heterocyclic ring, which may be substituted, including one or more nitrogen atoms attached to one or more carbon atoms such as to form the amino group and

(ii) those where —R⁷— comprises a carbocyclic or heterocyclic ring attached to the amino group, preferably the amino nitrogen, or spaced therefrom by no more than one carbon atom, preferably being directly attached to the-amino-group and preferably to its amino nitrogen

Preferred optional substituents for the heterocyclic or carbocyclic rings are selected from one or more of halo, amino, hydroxy, C₁₋₆ alkyl, C₁₋₆ hydroxyalkyl, C₁₋₆ alkoxy and C₇₋₁₂aralkyl.

B may be a single amino acid residue, an oligopeptide or a polypeptide. Where it is an oligopeptide it is typically of no more than 100 amino acid residues, eg. no more than 50, but more preferably from 1 to 10 amino acids and especially, eg. di, tri, tetra or penta-peptide. Most conveniently B is a single amino acid residue. The peptide group may contain spacer groups between the amino acids thereof. If present, such spacer groups are preferably selected from the same possibilities as group A and may alternate with the amino acid residues or otherwise replace all but key amino acids in a recognition sequence. B is preferably an α-amino acid or a peptide group made from α-amino acids. By “α amino acid”, we mean a compound such as those specified in U.S. Pat. No. 5,733,880, column 3, line 55 to column 4, line 39 incorporated herein by reference.

The di-, tri-, tetra-, penta-, oligo and polypeptides may be of any suitable amino acid sequence. One possible sequence (Suzuki (1989) EMBO J. 8, 797). (Ser-Pro-Lys-Lys)ₙ wherein n is 1 to 10, has been proposed as discussed in U.S. Pat. No. 5,733,880 and EP 0721447. Useful intermediates for synthesising such peptides are described in U.S. Pat. No. 5,733,880 column 4, lines 45 to 65. The syntheses of these compounds are described in detail in Bailly et al (1992) Anti-Cancer Drug Design (1992) 7, 83-100 incorporated herein by reference wherein the peptide was joined to the acridine heterocyclic ring system at the position opposite the N heteroatom in the middle ring.

By “isosteres” of the amino acids or peptides we include \acute{ω}-amino acids that have side chains that mimic the characteristic side chains of α-amino acid and peptides used in the invention. Examples of conventional isosteres are illustrated in ‘A Practical Guide to Combinatorial Chemistry, (1997) Edits. Czarnik and DeWitt, American Chemical Society ISBN 0-8412-3485-X, page 57, FIG. 2, eg. depsipeptides and peptoids, wherein the sidechains characteristic of α-amino acids are in alternative carried on ester group carbons or on amide group nitrogens; and in Medicinal Chemistry: Principles and Practice (1998) Edit: F D King, The Royal Society of Chemistry, ISBN-0-85186-494-5, Chapter 14, see Tables 2 page 208 re carboxylic amide groups in peptides; both incorporated herein by reference Also included are peptide mimics corresponding to peptides with amide bonds replaced by olefinic bonds.

By “derivatives” of the compounds of the invention, we include salts (acid or base addition), esters, amides, hydrazides and hydroxamic acids of the group B and other derivatives which do not diminish to an unacceptable extent the fundamental topoisomerase mediated activity, ie. anti-tumour properties of the compounds.

Salts which may be conveniently used in therapy include physiologically acceptable base salts, for example, derived from an appropriate base, such as an alkali metal (e.g. sodium), alkaline earth metal (e.g. magnesium) salts, ammonium and NX₄⁺ (wherein X is C₁₋₄ alkyl) salts. Physiologically acceptable acid salts include hydrochloride, sulphate, trifluoroacetate, mesylate, besylate, phosphate and glutamate.

Salts according to the invention may be prepared in conventional manner, for example by reaction of the parent compound with an appropriate base to form the corresponding base salt, or with an appropriate acid to form the corresponding acid salt.

Further preferred derivatives include those in which functional groups on the peptide group, which may be side groups or the terminal group, are capped. Suitable chemical groups to cap —NH— include —COCH₃, tertiary-butoxycarbonyl, benzyloxycarbonyl and other groups known in the art. Suitable chemical groups to cap —CO— include —OH or any —O-linked or —N-linked radical, for example —O-alkyl, —O-benzyl, —O-alkylaminoalkyl, —O-alkoxyalkyl or —NH—NHR⁹ where R⁹ is straight or branched alkyl, optionally substituted by —CN or —OH, an amide group (such as —CONH₂) and other groups known in the art. Examples of alkylaminoalkyl groups include CH₃(CH₃)NCH₂CH₂—, —(CH₂)₂NH(CH₂)₂OH and CH₃(CH₃)NCH₂CH₂NHCH₂CH₂—. Preferred capped —NH— groups are those where the side chain is capped as opposed to the terminal —NH₂. Where —X— is —C(O)— a preferred cap is —O—C₁₋₆, alkyl or —OH.

Where not otherwise defined, by “alkyl”, we include branched or straight chain alkyl of up to 20 carbon atoms, preferably 1-10 carbon atoms, more preferably 1-6 or 1-4 carbon atoms.

A useful discussion of alternative protective groups for amino acids (all types) and the scope of coupling reagents and deprotection reactions is to be found on pages 153-184 of a section called “chemical synthesis of peptides” in chapter 3 “Amino acids and Peptides” by R. S. Davidson and J. B. Hobbs in: “Natural Products, their Chemistry and Biological Significance”, Authors: J. Mann, R. S. Davidson, J. R. Hobbs, D. V. Banthorpe & J. B. Harbome, publ. Longman Scientific and Technical (1994), incorporated herein by reference.

It has been found that the compounds of the invention may be prepared as substantially pure optical isomers, ie. it is possible to synthesise them without inducing racemisation of chiral groups.

B is preferably the residue of an amino acid or oligopeptide and conveniently is the residue of an α-amino acid, but may be β-, γ-, δ-, ε-, ζ, η-amino acids where these are isosteres of peptides comprised of α-amino acids. For the avoidance of doubt, by the residue of an amino acid we mean the group which would remain after the carboxyl (—C(O)O—) functionality on the original amino acid has reacted to bond the amino acid to the —O-group of amino-R⁷—O— or the —NH— group of amino-R⁷—NH— or reacted to bond to the —C(O)— group of amino-R⁷—C(O)—, by way of an amide or ester bond, at the distal end of the spacer group A, to the anthracene ring moiety and in so doing become incorporated into the spacer group A, or a salt thereof. Thus, when B is the residue of an α-amino acid having the formula given above, it will have the formula:

HₙN⁺—CHR⁸—C(O)— or [X⁻]HₘN⁺—CHR⁸—C(O)—

—HN⁺—CHR⁸—C(O)—OH or —HN—CHR⁸—C(O)—O⁻[M^{+ or 2+}]

where R⁸ is a characteristic group of such acid, eg such as specified as in U.S. Pat. No. 5,733,880 for R⁷ incorporated herein by reference, and n is 1 or 2 m is 2 or 3 and X is or M^{+ or 2+} are counter ions.

Preferably B comprises one or more independently selected residues of alanine, phenylalanine, glycine, proline, valine, leucine, methionine or tyrosine and naturally or non-naturally occurring amino acids and analogues of similar charge and hydrophobicity or hydrophilicity. For example, instead of including phenylalanine, an amino acid or oligopeptide that is or includes a halo- or alkoxy or alkylthio group substituted -phenylalanine or -phenylglycine is preferred for treating some forms of topoisomerase I expressing tumour, eg L- or D-4-chlorophenylalanine or L- or D-4-chlorophenylglycine or L- or D4-methoxytyrosine.

Particularly preferred residues are of amino acids and peptides the internal amide bonds or the ester bond to -amino-R⁷— of which that are resistant to degredation by enzymes in vivo. For example use of D-amino acids or N-alkylated amino acids such as N-methylglycine (sar), N-methylalanine. More preferred are di-, tri- and tetra-peptides. The L-isomer is usually preferred in each case, although D-isomers may be preferred, eg D-Phe.

Preferred groups Y include a hydrogen atom and alkyl, aryl, aralkyl, e.g. benzyl, and acyl, e.g. tert-butoxy-carbonyl, groups.

In one preferred embodiment, R¹ and R² are both H, R³ and R⁴ are both oxo, R⁵ is a group —A—B and R⁶ is H. In another preferred embodiment, R¹ is OH, R² is H, R³ and R⁴ are both oxo, R⁵ is a group —A—B and R⁶ is OH. In yet another preferred embodiment, R¹ and R² are both H, R³ and R⁴ are both oxo, R⁵ is a group —A—B and R⁶ is OH. In a further preferred embodiment, R¹ and R² are both OH, R³ and R⁴ are both oxo, R⁵ is a group —A—B and R⁶ is OH.

Preferred compounds for the use for the invention are of formula III

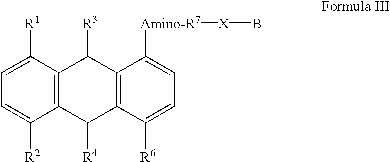

characterised in that amino is a group selected from NC₄, NC₅, N₂C₃ and N₂C₄ heterocyclic rings, ie. pyrrole, 2H-pyrrole, pyrrolidine, pyrroline, imidazole, imidazidine, imidazoline, pyrazole, pyrazolidine, pyrazoline, pyridine, pyrazine, piperidine, and piperazine. —R⁷— may be bonded to any of the atoms of the moiety completing the ring and amino is bonded to the anthraquinone ring directly through one of the nitrogens. Most preferably X is —NH— or —C(O)—. The heterocyclic ring is preferably saturated.

A further preferred group of compounds of formula I, II or III are those where amino is —NH— or —NR¹⁰— and R⁷ incorporates the carbocylic or heterocylic ring, which is in turn spaced from the amino nitrogen by no more than two carbon atoms, preferably being spaced by no more than one carbon atom, and most preferably being directly attached to the amino nitrogen.

Most preferred compounds have groups A as described in the formulae of FIG. 1 attached hereto or are derived from anthraquinone-A-intermediates which as shown therein.

A second aspect of the present invention provides novel compounds of formula I that have use in the first aspect of the present invention. These compounds are those of formula II, and preferred ones are as described for the use of the invention, being those that are not disclosed in copending PCT/GB99/01901 or U.S. Pat. No. 5,733,880.

Thus, the second aspect of the present invention provides compounds of formula IV

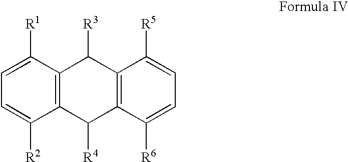

wherein

at least one of R¹, R², R⁵ and R⁶ is a group —AB and the others are independently selected from hydrogen, hydroxy, alkoxy, acyloxy, a group —AB and a group -amino-(R⁷)ₙX—Y wherein R⁷ is a divalent organic radical and n is 0 or 1;

R³ and R⁴ are independently oxo, hydroxy or hydrogen;

the or each A is independently a spacer group of formula -amino-(R⁷)ₙ—X— which is bonded to the anthracene ring via the amino group nitrogen and to B via —X—

X is independently selected from O, NH and C(O);

B is an independently selected amino acid residue or a peptide group or isostere thereof; and

Y is hydrogen or a capping group,

characterised in that the group -amino-R⁷—X— incorporates one or more optionally substituted carbocyclic, or heterocylic rings and is selected from

(i) those groups where -amino- comprises a heterocyclic ring including one or more nitrogen atoms attached to one or more carbon atoms such as to form the amino group and

(ii) those groups where —R⁷— comprises a carbocyclic or heterocyclic ring attached to the amino group, preferably the amino nitrogen, or spaced from the amino nitrogen by no more than four carbon atoms, preferably two or less, preferably being directly attached to the -amino-group attached to the anthraquinone ring and preferably to its amino nitrogen

Most preferably —X— is —NH—

A third aspect of the present invention provides a process for preparing a compound of formula IV comprising:

(A) reacting a compound of formula V

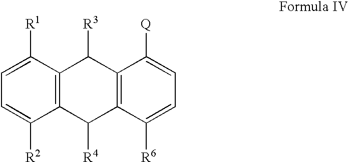

where R¹ to R⁴ and R⁶ are independently selected from those group as defined for the first aspect and a group Q, wherein Q is a reactive group such as —Cl, —Br or —OH, with an amino acid or diamine, e.g. an αω-diaminoalkane, to form a compound having the formula V:

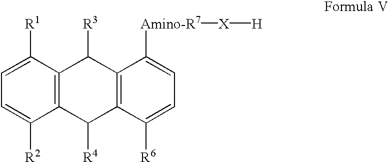

wherein when R¹ to R⁶ were reactive groups they may also be independently selected amino-R⁷—X—H in Formula V.

and (B) reacting the compound of Formula V with an amino acid or peptide or isostere to give a compound of Formula I.

The corresponding compounds of formula I where —X— is —O— may be prepared by using an ωamino alkanol as required or a compound where a hydroxy is included in —R⁷— as disclosed in PCT/GB99/01901.

Compounds of Formula IV in which Q is Cl or Br and both R³ and R⁴ are oxo are commercially available. The reaction generally proceeds in an aprotic solvent (e.g. DMSO or DMF). One compound of the invention can be converted to another by, for example, oxidising —H at R¹ and/or R² to —OH; oxidising —H at R³ and/or R⁴ to —OH; oxidising —OH at R³ and/or R⁴ to oxo, for example in an aerial oxidation or using chloranil; or reducing oxo at R³ and/or R⁴ to —OH (for example with sodium dithionite or zinc/acetic acid) or onward to —H. The sodium dithionite reaction is described in Marschalk et al (1936) Bull. Soc. Chim. Fr. 3, 1545, and the Zn/CH₃COOH reaction in Morris, G. A. et al (1986) Tetrahedron 42, 3303 both incorporated herein by reference. Another conversion of one compound of the invention to another involves extending the B group by removing any cap which is present and adding one or more amino acid residues.

Compounds where all of R¹, R², R⁵ and R⁶ are reactive groups are described in Katzhandler et al (1989) Eur. J. Med. Chem 24 p23-30 and R K Y Zee-Cheng et al (1987) J. Med. Chem 30, p1682-1686, both of which are incorporated herein by reference.

Prior to step (B), the amino group of the amino acid should be protected by a group such as tertiary-butyloxycarbonyl, benzyloxycarbonyl, fluorenylmethoxycarbonyl, and the like, to avoid interference during condensation with the anthracene compound eg. the anthraquinone. Similarly, those amino acids which contain functionality in their side-chains in general also need to have the functionality protected. The protecting groups used on the side chain can be the same or different than those used to protect the amino radical. The protecting group can be removed after step (B) has been completed. Methods for applying and removing protecting groups are taught in U.S. Pat. No. 5,733,880, column 9, line 9 to column 10, line 20. U.S. Pat. No. 5,733,880 is incorporated herein by reference for all the referenced techniques and definitions for which it is referred to above.

In a further aspect the invention provides a pharmaceutical preparation comprising a pharmaceutically acceptable carrier and/or excipient and a compound of the first or second aspect. Any suitable pharmaceutically acceptable carrier can be used. The preparation should be suitable for administration in the chosen manner. In particular, it should be sterile and, if intended for injection, non-pyrogenic.

Administration of the aforementioned compounds of the invention or a formulation thereof need not be restricted by route. Options include enteral (for example oral and rectal) or parenteral (for example delivery into the nose or lung or injection into the veins, arteries, brain, spine, bladder, peritoneum, muscles or subcutaneous region. The compounds may be injected directly into the tumour. The treatment may consist of a single dose or a plurality of doses over a period of time. The dosage will preferably be determined by the physician but may be between 0.01 mg and 1.0 g/kg/day, for example between 0.1 and 500 mg/kg/day. In terms of dose per square meter of body surface, the compound can be administered at 1.0 mg to 1.5 g per m² per day, for example 3.0-200.0 mg/m²/day. At least some compounds of the invention have a particularly low toxicity to normal mammalian cells and could be given in quite high doses, for example 50-300 mg/kg. By comparison doxorubicin has a maximum tolerated dose of 5 mg/kg in rodents and 1-2 mg/kg in man.

Whilst it is possible for a compound of the invention to be administered alone, it is preferable to present it as a pharmaceutical formulation, together with one or more acceptable carriers and/or excipients. The carrier(s) and/or excipients must be “acceptable” in the sense of being compatible with the compound of the invention and not deleterious to the recipients thereof.

The formulations may conveniently be presented in unit dosage form and may be prepared by any of the methods well known in the art of pharmacy. A unit dosage form may comprise 2.0 mg to 2.0 g, for example 5.0 mg to 300.0 mg of active ingredient. Such methods include the step of bringing into association the active ingredient, ie. the compound of the invention, with the carrier and/or excipients which constitute one or more accessory ingredients. In general the formulations are prepared by uniformly and intimately bringing into association the active ingredient with liquid carriers or finely divided solid carriers and/or excipients and/or two or all of these, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, PVP, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which may render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

At least some of the compounds are useful as anticancer, antiviral and/or antiparasitic drugs and at least some of the anticancer compounds can be used against most malignancies.

Particular tumours suitable for treatment in accordance with the invention include leukaemias, and cancers of the uterine cervix, head, neck, brain gliomas, breast, colon, lung, prostate, skin, mouth, nose, oesophagus, stomach, liver, pancreas and metastatic forms of any of these.

Particular viral infections suitable for treatment in accordance with the invention include those caused by the viruses herpes simplex virus I (HSV I); herpes simplex virus II (HSV II); varicella-zoster virus/Ellen (VZV Ellen); human papilloma virus (HPV); and human immunodeficiency virus (HIV).

Particular protozoal infections suitable for treatment in accordance with the invention include trichomoniasis; malaria (especially that caused by Plasmodium falciparum); trypanosomiasis (caused by Trypanosoma brucei and T. cruzi); and leishmaniasis. It will be appreciated by those skilled in the art that the novel profile of activity of the present compounds will make some at least useful as antibacterial agents.

A further aspect of the present invention provides a method of treating a human or animal body in need of therapy for a disorder selected from the group consisting of cancer, viral infection or parasitic infection comprising administering to said human or animal body an effective therapeutic dose of a compound or preparation of the invention.

The invention will now be described by way of illustration only by way of the following Examples, Tables and Figures.

### FIGURES

FIG. 1 shows examples of the moiety anthraquinone-amino-R⁷—X— compound intermediates of the invention that are suitable for processing to link to group B and amino acid and peptide conjugates of these which are the active compounds of the invention. Some of these intermediates are active, at least in vitro, toward Topo I. The compound for reaction with chloroanthraquinone in synthesis is also given.

FIG. 2 shows the results of an immunoband depletion experiment evidencing depeltion of Topoisomerase I under action of control compounds and a preferred compound of the invention.

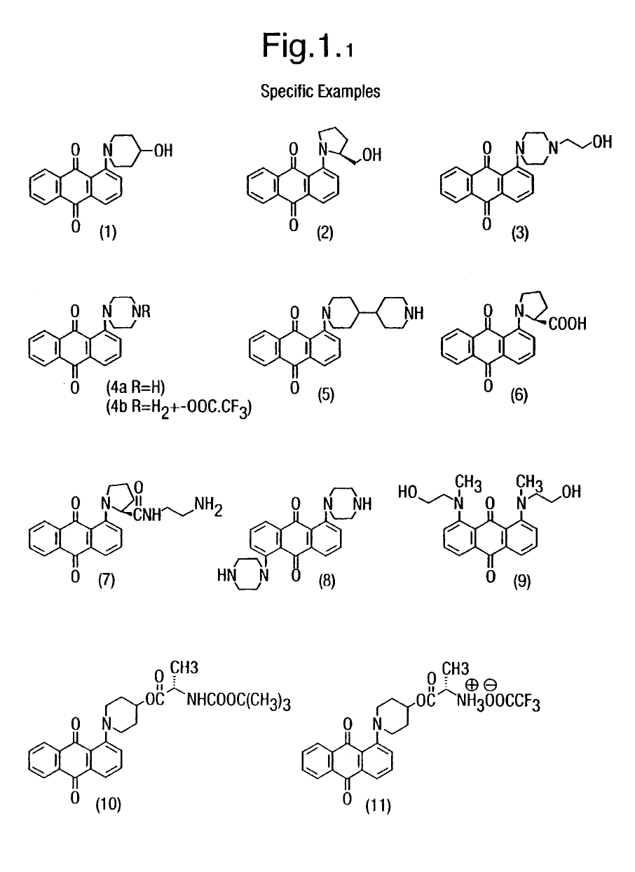

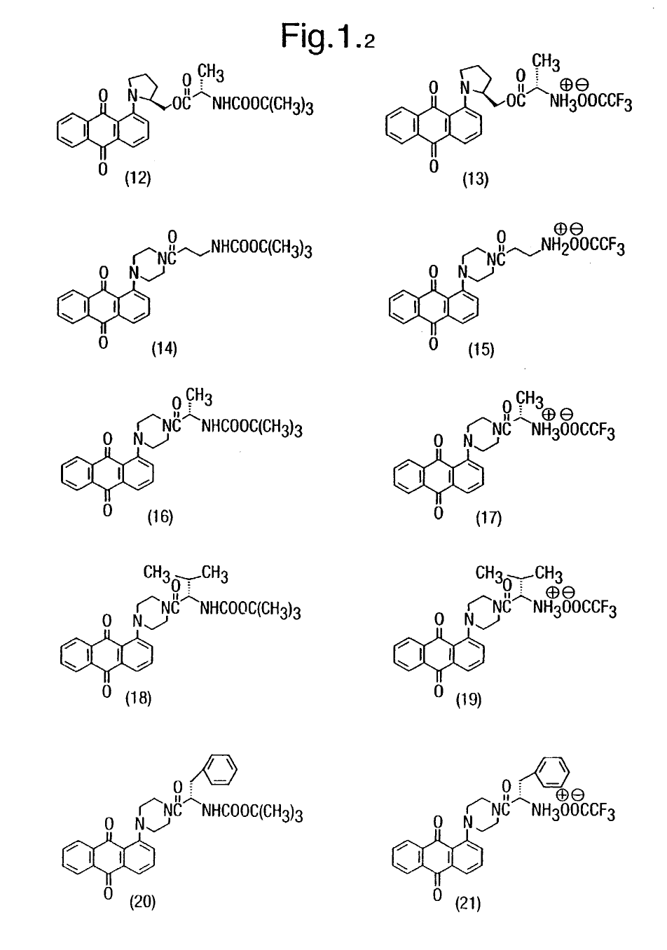

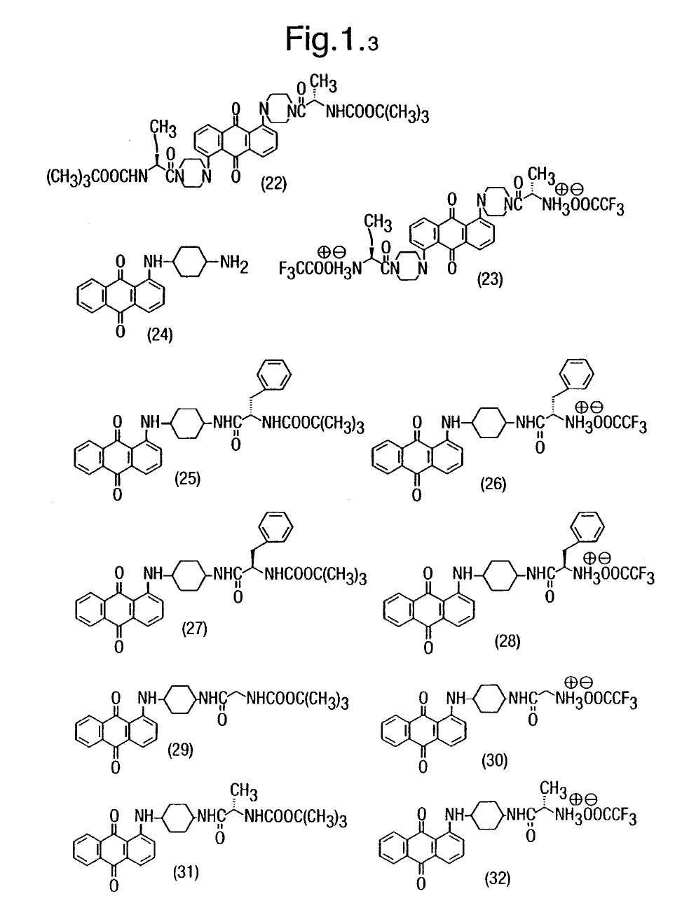

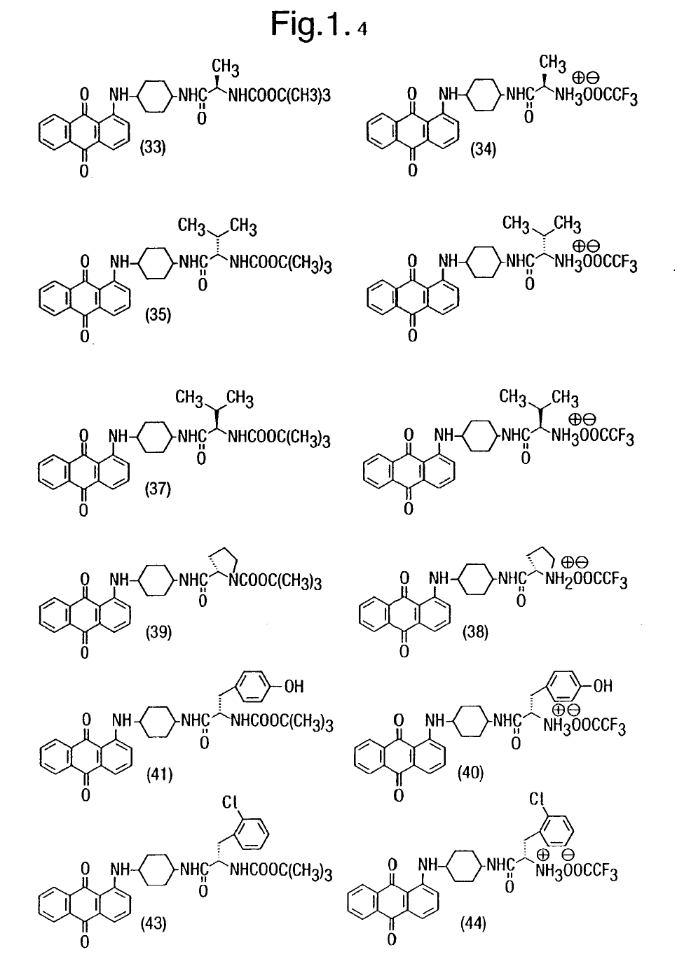

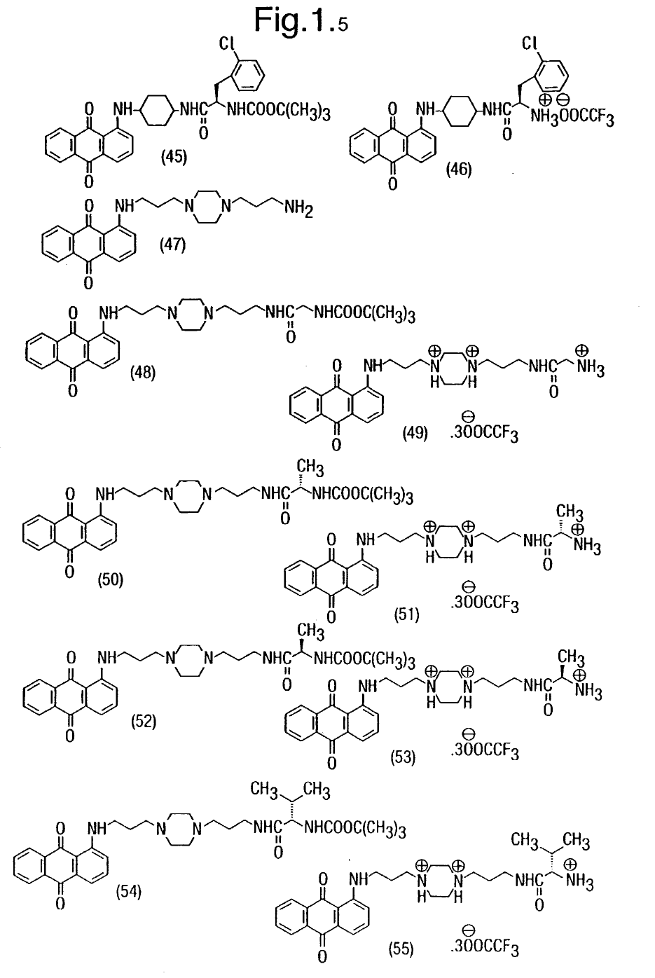

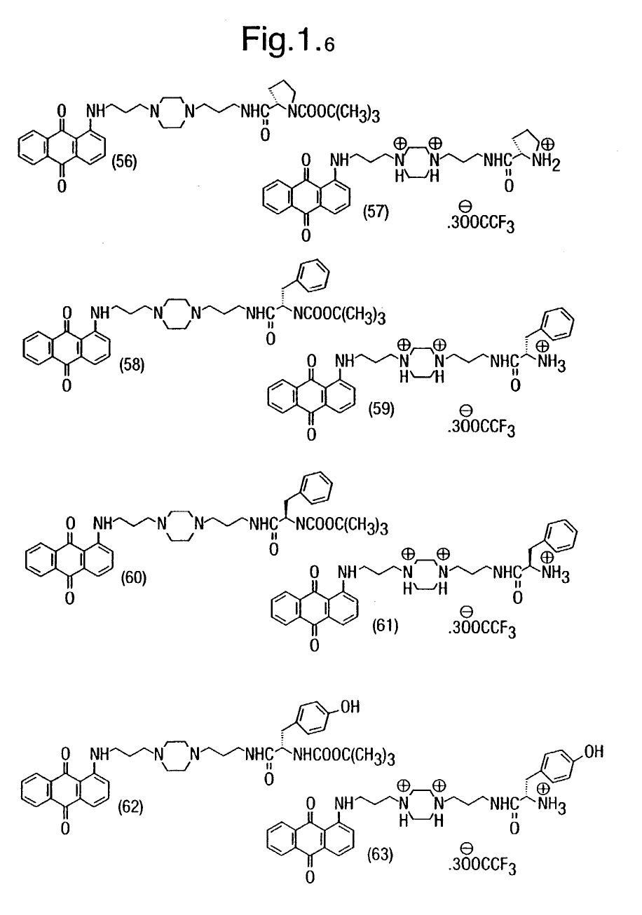

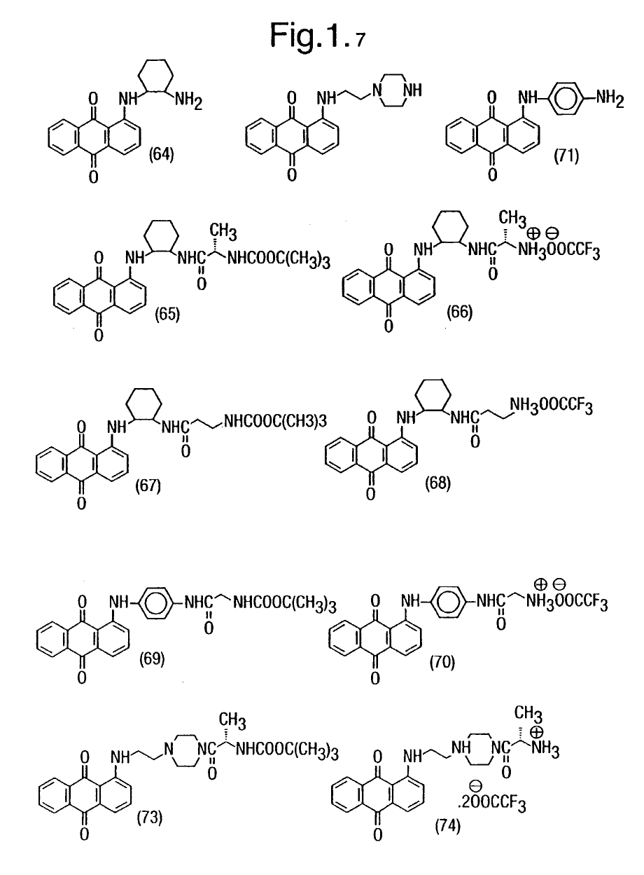

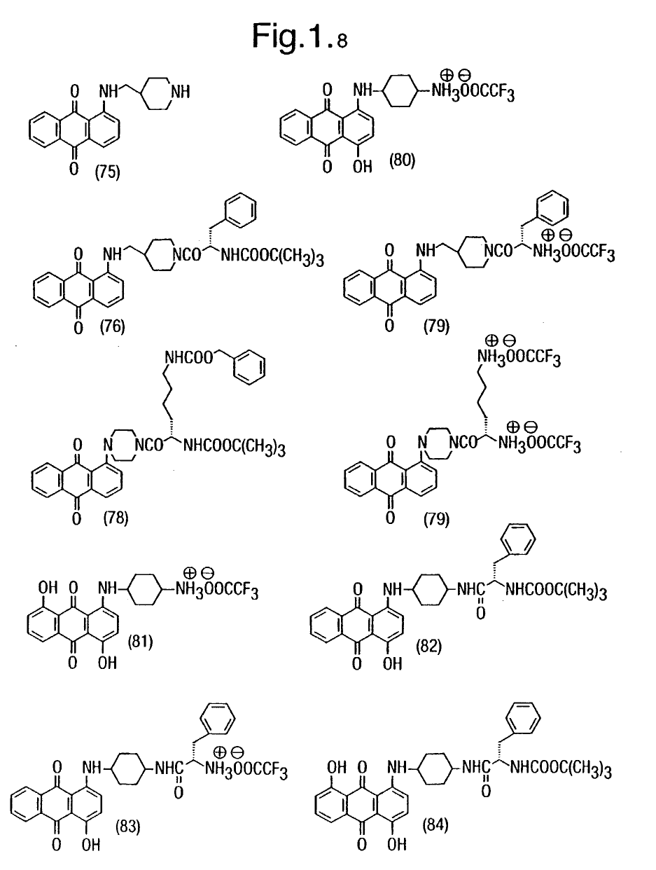

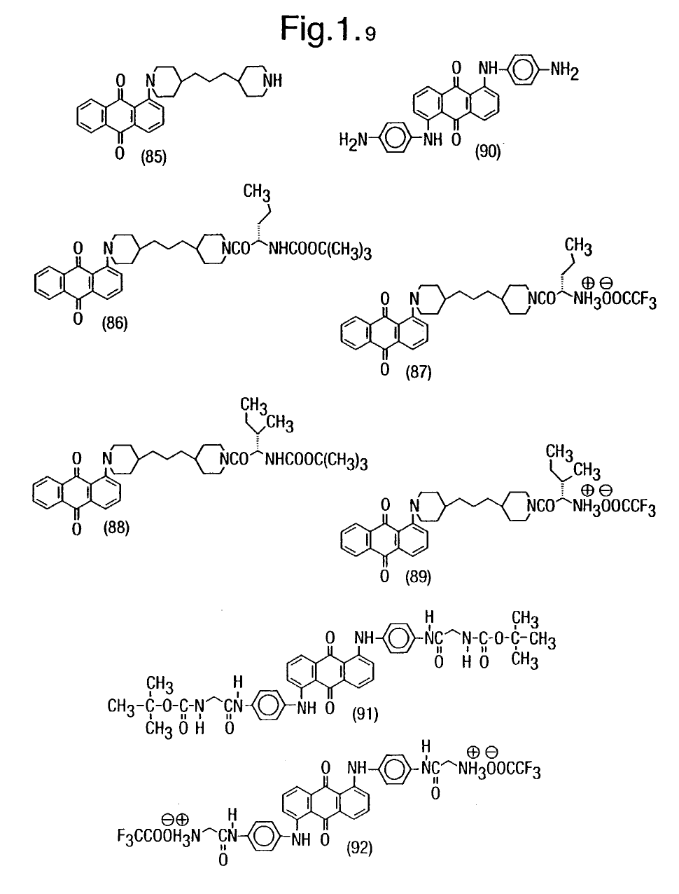

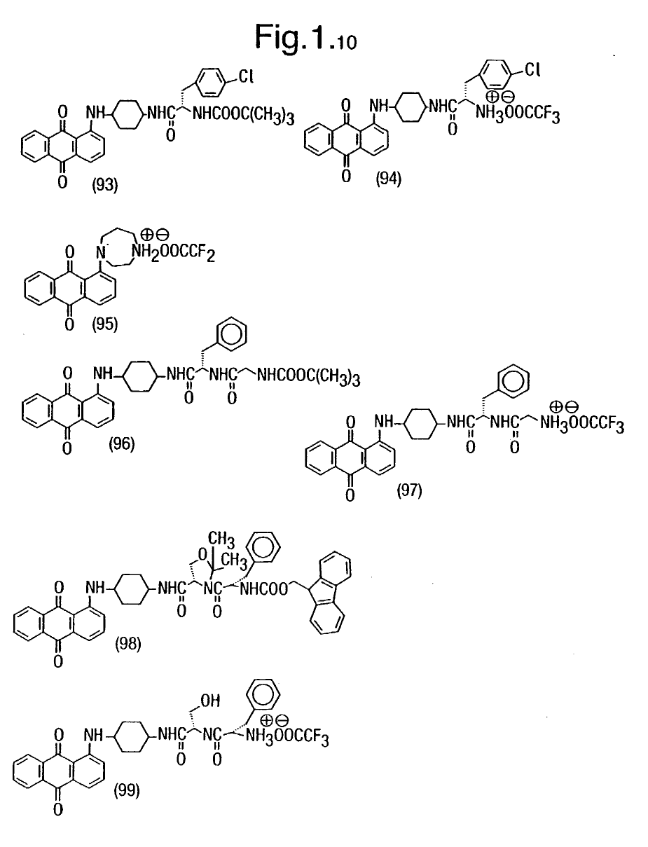

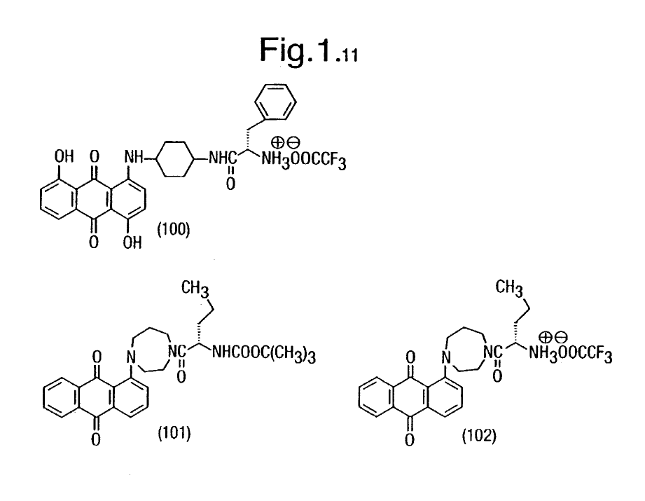

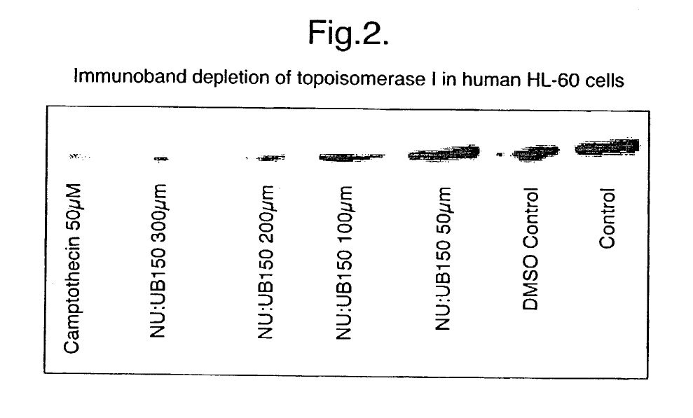

The following specific Examples illustrate preferred, non-limiting compounds and processes of the invention. Further examples falling within the scope of the claims will occur to those in the light of these. Methods 1 to 9 are provided to illustrate the general methods for obtaining variously substituted anthracene ring compounds with amino-R⁷—X— spacers attached thereto at least at the 1-position. Examples 1 to 102 describe preparation of specific anthraquinones, intermediates derived therefrom with a variety of spacers at at least the 1-position and preparation of anthraquinones derivatives of the invention possessing the spacer group -amino R⁷—X— linked by the amino —N— to the 1-position and by—X— to amino acids and peptides. These methods can also be used with isosteres such as peptoids and despeptides wherein the amino acid and peptides are replaced by said peptoids and despeptides in the synthesis.

NMR data is provided for certain key compounds but where it is not given this is for reason of brevity; NMRs obtained for examples are consistent with the structures described and shown in the Tables and Figures.

Note: where hydroxychloroanthroquinones are used as starting materials, pyridine may be used as solvent to reduce alternative replacement of hydroxy groups by the amine in each case.

In examples where the secondary amine used contains a free carboxylic acid function, the sodium salt of the acid is employed. Consequently the reaction mixture contains an appropriate amount of water to maintain the sodium salt in solution and the anthraquinone produced is isolated by pouring into hydrochloric acid (500 ml, 5M) with ice cooling.

Where bis-A- compounds are being produced the secondary amine is added in a ratio 500 mmol to 10 mmol dichloroanthraquinone in 15 ml of the solvent. Appropriate adjustment of concentrations is made when preparing the tris-A or tetrakis-A compounds.

Anthraquinone Spacer Compounds

Aminated Derivatives

The aminated anthraquinones (spacer compounds) may conveniently be prepared by nucleophilic displacement of chlorine (or other suitable halogen) from a mono-chloro- (or halo)anthraquinone by an appropriate amine. Monochlorinated anthraquinones, for example, 1-, and 2-chloroanthraquinone, are either commercially available or easily prepared by published procedures [J. Katzhendler, K. F. Gean, G. Bar-Ad, Z. Tashma, R. Ben-Snoshan, I. Ringel, U. Backrack, Eur. J. Med. Chem, (1989), 24, 23.] which include chloro-hydroxyanthraquinones; pyridine is used as a solvent to prevent or minimise alternative replacement of hydroxy groups by the amine whereas selective replacement of hydroxy groups occurs when the solvent is butanol. Judicious choice of solvent thus allows sequential selective aminations to be regioselective for mono-, di-, tri- and tetra-aminated substitution patterns. Additionally, the amino substituents may selectively be identical or non-identical. Fluorinated anthraquinones also provide convenient intermediates for the regiospecific introduction of amino-substituents into the anthraquinone nucleus thus affording alternative syntheses of the spacer compounds of this invention. [A. P. Krapcho, Z. Getahun, K. L. Avery, Jr. K. J. Vargus, M. P. Hacker, S. Spinelli, G. Pezzoni and C. Manzotti, J. Med. Chem., (1991), 34, 2373].

It is thus possible to introduce regiospecifically two, three or four aminoalkyl residues into the anthraquinone nucleus. Mono-aminated derivatives or 1,4-; 1,5-; 1,8-; and 2,6-BIS-aminated substitution patterns are preferred

Synthesis of Anthraquinone Spacer Compounds

Method 1: General Method for the Preparation of Mono-aminated Anthraquinone-spacer Arm Compounds from Secondary Amines.

The appropriate (unsubstituted or substituted) monochloroanthraquinone (10 mmol) was suspended in DMSO (15 cm³); an appropriate secondary amine (350 mmol) was added and the mixture was heated for 1 h over a boiling water bath (or heated at reflux as appropriate). The solution was cooled and added to a large excess of water (500 cm³). The red precipitated solid was filtered off, dried and could be used for subsequent reactions without further purification. Analytically pure samples were obtained by column chromatographic purification on silica gel 60[Merck] using chloroform: methanol and gradient elution.

note. In examples where the secondary amine also contained a free carboxylic acid function, the sodium salt of the acid was employed. Consequently, the reaction mixture contained an appropriate volume of water to maintain the sodium salt in solution and the anthraquinone product was isolated by pouring into hydrochloric acid (500 cm³, 5M), with ice-cooling.

note. In examples in which the monochloroanthraquinone additionally contained hydroxyl groups, pyridine was used in place of DMSO.

Method 2: General Method for the Preparation of 1,4- 1,5- 2,6- or 1,8-Bis-Aminated Anthraquinone Spacer Arm Compounds.

The appropriate (unsubstituted or substituted) dichloroanthraquinone (10 mmol) was suspended in DMSO (15 cm³); the appropriate primary or secondary amine (500 mmol) was added and the mixture was heated at reflux for 1-2 h. After cooling, the mixture was added to a large volume of water (500 cm³). The red (to purple) solid was filtered off, dried and could be used for subsequent reactions without further purification.

note. In examples of dichloroanthraquinones which additionally contained hydroxyl groups, pyridine was used in place of DMSO.

Method 3: General Method for the Preparation of ‘Anthraquinone-spacer Arm’ Compounds Used in the Preparation of Carbocyclic Ring Spacers.

The appropriate (unsubstituted or substituted) mono-chloroanthraquinone (40 mmol) was suspended in DMSO (15 cm³); an α,ω-diaminoalkane (200 mmol) was added and the mixture was heated for 0.5 h over a boiling water bath. The solution was cooled and added to a large excess of water (500 cm³). The red precipitated solid was filtered off, dried and used for subsequent reactions without further purification.

Synthesis of Spacer-Linked Anthraquinone Amino Acid (Peptide) Conjugates

Anthraquinone-Spacer-Amide Linked-Amino Acid (Peptide) Conjugates

Method 4: General Method for Coupling of a N-α-protected-C-activated Amino Acid to a Pre-formed Anthraquinone-aminoalkylamino Spacer Compound

The (aminoalkylamino)anthracene-9,10-dione spacer compound (3.0 mmol) was suspended in DMF (70 cm³) and stirred at 0° C. An N-α-protected amino acid-O-pentafluorophenolate ester (3.3 mmol) in DMF (30 cm³) [or an N-α-protected amino acid-N-hydroxysuccinimide active ester (3.3 mmol) in THF (30 cm³)] was added—dropwise and the reaction mixture was allowed to reach room temperature. Stirring was continued for a further 12 h. The solution was concentrated by partial evaporation of the solvent, then the mixture was partitioned between chloroform and water. The chloroform extracts were washed with saturated sodium bicarbonate_{(aq)}, then water, dried (MgSO₄), filtered and evaporated to a low volume (10 cm³). The foregoing concentrated solution was then applied to a silica gel chromatography column (4×30 cm) prepared with chloroform:ethyl acetate, 4:1 and eluted initially in the same solvent mixture to remove a little coloured highly mobile impurity. The major product was eluted using the same solvent mixture containing methanol (2% v/v). Fractions containing the major product were combined, filtered and evaporated to give a red solid. Recrystallisation from ethanol (or appropriate alternative) afforded the (N-protected) spacer-linked anthraquinone amino acid conjugate in an analytically pure form.

Anthraquinone-Spacer-Ester Linked-Amino Acid (Peptide) Conjugates

Method 5: General Method for Coupling of a N-α-protected Amino Acid to a Pre-formed Anthraquinone-hydroxyalkylamino Spacer Compound.

Dicyclohexylcarbodiimide (DCC) (3.3 mmol) and 4-dimethylaminopyridine (DMAP) (0.15 mmol) in dichloromethane (35 cm³) was added to a cooled stirred solution of an (hydroxyalkylamino)anthracene-9,10-dione (3 mmol) and a N-α-^{t}Boc-protected amino acid (3.3 mmol) in dichloromethane (35 cm³). Stirring was continued for 12 h as the mixture was allowed to reach room temperature. The precipitated dicyclohexylurea (DCU) was filtered off and the solution partitioned between chloroform and water (1:1, 100 cm³), washed three times with water (50 cm³), dried (MgSO₄), filtered and evaporated to dryness. The solid was dissolved in toluene, applied to a silica gel column and eluted with increasing gradients of toluene/ethyl acetate. Recrystallisation from a suitable solvent afforded the spacer-linked anthraquinone (N-protected) amino acid conjugate in an analytically pure form.

Method 6: General Method for the Deprotection of N-tertiarybutoxycarbonyl (^{t}Boc) Protected Anthraquinone Spacer (Ester or Amide) Linked Amino Acid Conjugates.

The ^{t}Boc protected compound (3 mmol) was dissolved in trifluoroacetic acid (7 cm³) at room temperature. After 20 minutes the solvent was evaporated and the solid re-evaporated with ethanol (3×10 cm³) before dissolving in a minimum volume of ethanol (3 cm³). Addition of ether (100 cm³) gave a precipitate of the deprotected anthraquinone spacer-linked amino acid conjugate as the trifluoroacetate salt which was filtered off and dried.

Specific Method: Method 7 Preparation of 1-[(H—X—R⁷-amino]anthracene-9,10-dione

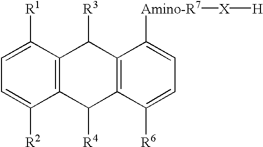

Note, for all methods 7 to 11, where —X— is —C(O)— then HO—X— applies to title compound.

The method described below was used to prepare a number of compounds of the above formula in which the nature and length of the chain of atoms separating the -amino- group from the —X— atom was varied.

1-chloroanthraquinone (10 mM) was suspended in DMSO (5 cm³) and an ω-amino-R⁷-alkanol, ω-cyclicamino-R⁷-alcohol, ω-amino acid, ω-cyclicamino-R⁷-acid, αω—R⁷-diamine or acyclicamino-ω—R⁷-amine (350 mM) was then added. The mixture was heated for 1 hour over a boiling water bath or at reflux for 2 hours as appropriate, cooled and then added to a large excess of water (500 cm³). The red precipitated solid of 1-[(H—X—R⁷-amino]anthracene-9,10-dione was filtered off and recrystallised from ethanol.

Specific method: Method 8: Preparation of 4-hydroxy-1-[(H—X—R⁷-amino]anthracene-9,10-dione

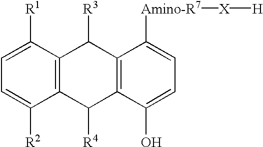

The method described below was used to prepare a number of compounds of the above formula in which the length of the chain of atoms separating the amino group from the —X— atom was varied.

1,4-dihydroxyanthraquinone (10 mM) and an ω-amino-R⁷-alkanol, cyclicamino-R⁷-alcohol ω-amino-R⁷-acid, ω-cyclicamino-R⁷-acid, αω—R⁷-diamine or acyclicamino-ω—R⁷-amine (120 mM) were suspended in ethanol (50 cm³) and THF (50 cm³) and heated over a water bath at 95° C. for 1.75 hours. The solution was cooled and immediately applied to a silica gel chromatography column using toluene/ethyl acetate as the eluting solvent to give 4-hydroxy-1-[(hydroxy-R⁷)amino]anthracene-9,10-dione as a purple solid after recrystallisation from ethanol.

Specific method: Method 9 Preparation of 4,8-dihydroxy-1-[(H—X—R⁷amino]anthracene-9,10-dione

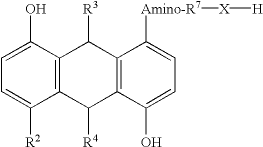

The method described below was used to prepare a number of compounds of the above formula in which the length of the chain separating the amino group from the —O— group was varied.

Leuco-1,4,5-trihydroxyanthraquinone (4 mM) was dissolved in dichloromethane (250 cm³) at room temperature, under nitrogen and an ω-aminoalkanol (4 mM) was then added. The reaction was stirred for 24 hours at room temperature. At the end of this period, triethylamine (0.5 cm³) was added and the solution was aerated for 2 hours whereupon the colour changed from green to purple. The solvent was evaporated to a low volume and was applied to a silica-gel chromatography column and eluted with dichloromethane with an increasing gradient of toluene-ethyl acetate (4:1). Fractions containing the major products were combined, filtered and evaporated to dryness and the residue was recrystallised from ethanol to give the title compound.

### EXAMPLE (1)

### 1-(4-hydroxypiperidyl)anthracene-9,10-dione. [Method 1]

Prepared using 4-hydroxypiperidine and 1-chloroanthraquinone. Mp 145° C. CIMS(+) m/z: 308 (10%)(MH)⁺, 260 (100%), M, 307.

### EXAMPLE (2)

### 1-[(2S)-2-(hydroxymethyl)pyrrolidinyl]anthracene-9,10-dinone. [Method 1]

Prepared using L-prolinol, 1-chloroanthraquinone and pyridine (1 eq). Mp 134° C. FABMS(+) m/z: 330 (15%)(M+Na)⁺, 308 (100%)(MH)⁺. M, 307.

### EXAMPLE (3)

### 1-[4-(2-hydroxyethyl)piperazinyl)anthracene-9,10-dione. [Method 1]

Prepared using 1-(2-hydroxyethyl)piperazine and 1-chloroanthraquinone. Mp 140° C. FABMS(+) mass spectrum had m/z 337 (100%)(MH)⁺. M, 336.

### EXAMPLE (4a)

### 1-(1-piperazinyl)anthracene-9,10-dione. [Method 1]

Prepared using piperazine hexahydrate and 1-chloroanthraquinone. Mp 190° C. CIMS(+) m/z: 293 (100%)(MH)⁺, 279 (25%), 87 (60%). M, 292.

### EXAMPLE (4b)

### 1-(1-piperazinyl)anthracene-9,10-dione trifluoroacetate salt

Example (4a) was dissolved in trifluoroacetic acid and evaporated to dryness. The residue was recrystallised from ethanol/diethyl ether. Mp 170° C. ESMS(+) m/z: 293 (100%)(RNH₃)⁺, 232 (70%). ESMS(−) m/z: 113 (95%), 69 (100%). M, 406.

### EXAMPLE (5)

### 1-[4-(4-piperidinyl)piperidinyl]anthracene-9,10-dione. [Method 1]

Prepared using bipiperidine and 1-chloroanthraquinone. Mp 155-160° C. CIMS(+) m/z: 397 (3%), 375 (100%)(MH)⁺. M, 374.

### EXAMPLE (6)

### (2S)-1-(9,10-dioxoanthryl)prrolidine-2-carboxylic acid. [Method 1]

Prepared using L-proline and 1-chloroanthraquinone. Mp 175-185° C. FABMS(+) m/z: 322 (100%)(MH)⁺. M, 321.

### EXAMPLE (7)

### [(2S)-1-(9,10-dioxoanthryl)pyrrolidin-2-yl]-N-(2-aminoethyl)carboxamide. [Method 4]

(2S)-1-(9,10-Dioxoanthryl)pyrrolidine-2-carboxylic acid (6) (1 eq) was converted to its pentafluorophenolate ester [compound (6) 1 eq, pentafluorophenol 1.1 eq and dicyclohexylcarbodiimide 1.2 eq in ethyl acetate at 0° C. for 3 h] and reacted with N-^{t}Boc-1,2-diaminoethane. The N-^{t}Boc conjugate was deprotected using trifluoroacetic acid [Method 6]. Addition of triethylamine and extraction into chloroform afforded the title compound. Mp 164-170° C. FABMS(+) m/z: 367 (100%)(MH)⁺. M, 363.

### EXAMPLE (8)

### 1,5-dipiperazinyl anthracene-9,10-dione. [Method 2]

Prepared using piperazine hexahydrate and 1,5-dichloroanthraquinone. CIMS(+) m/z: 377 (100%)(MH)⁺. M, 376.

### EXAMPLE (9)

### 1,8-bis[(2-hydroxyethyl)methylamino]anthracene-9,10-dione. [Method 2]

Prepared using 2-(methylamino)ethanol and 1,8-dichloroanthraquinone. Mp 168-178° C. FABMS(+) m/z: 377 (5%), 355 (100%)(MH)⁺. M, 354.

### EXAMPLE (10)

### 1-(9,10-dioxoanthryl)-4-piperidyl-(2S)-[(tert-butoxy)carbonylamino]-propanoate [Method 5]

Prepared from anthraquinone-spacer compound (1) and N-tertiarybutoxycarbonyl-L-alanine. Mp 98° C. FABMS(+) m/z: 501 (16%)(M+Na)⁺, 479 (100%) (MH)⁺. M, 478.

### EXAMPLE (11)

### 1-(9,10-dioxoanthryl)-4-piperidyl-(2S)-2-aminopropanoate trifluoroacetate salt. [Method 6]

Prepared by deprotection of example (10). Mp 102° C. ESMS(+) m/z: 379 (100%)(RNH₃)⁺. ESMS(−) m/z: 113. M, 492. ¹H nmr spectrum (d₆-DMSO)(200 MHz) δ: 1.48 (3H, d); 1.95 (2H, m); 2.10 (2H, m); 3.15 (2H, m); 3.35 (2H, m); 4.15 (1H, q); 5.05 (1H, qn); 7.55 (1H, dd); 7.75 (2H, m); 7.85 (2H, m); 8.15 (2H, m); 8.40 (3H, br.s).

### EXAMPLE (12)

### [(2S)]-1-(9,10-dioxoanthryl)pyrrolidin-2-yl]methyl (2S)-2-[(tert-butoxy)carbonylamino]propanoate. [Method 5]

Prepared from anthraquinone-spacer compound (2) and N-tertiarybutoxycarbonyl-L-alanine. Mp 110° C. FABMS(+) m/z: 501 (15%)(M+Na)⁺, 479 (100%)(MH)⁺. M, 478. ¹H nmr spectrum (CDCl₃)(200 MHz) δ: 1.25 (3H, d); 1.45 (9H, s); 1.75 (1H, m); 2.0 (2H, m); 2.35 (1H, m); 2.55 (1H, dd); 3.80 (1H, m); 4.20-4.50 (4H, m); 5.40 (1H, d); 7.55 (12H, m); 7.75 (3H, m); 8.20 (2H, m). C₂₇H₃₀N₂O₆ requires C, 67.8; H, 6.3; N, 5.9%. Found C, 68.3; H, 6.0; N, 5.8%.

### EXAMPLE (13)

### [(2S)]-1-(9,10-dioxoanthryl)pyrrolidin-2-yl]methyl (2S)-2-aminopropanoate trifluoroacetate salt. [Method 6]

Prepared by deprotection of example (12). Mp 130° C. ESMS (+) m/z: 379 (38%)(RNH₃)⁺. ESMS(−) m/z: 113 (100%). C₂₄H₂₃N₂O₆F₃ requires C, 58.5; H, 4.7; N, 5.7%. Found C, 58.4; H, 4.4; N, 5.6%.

### EXAMPLE (14)

### 1-(1-N-tertiarybutoxycarbonyl-β-alanylpiperazinyl)anthracene-9,10-dione. [Method 4]

Prepared from anthraquinone-spacer compound (4a) and N-tertiarybutoxycarbonyl-β-alanine N-hydroxysuccinimide ester. Mp 185° C. CIMS(+) m/z: 464 (65%)(MH)⁺, 364 (50%), 158 (100%). M, 463.

### EXAMPLE (15)

### 1-(2-β-alanylpiperazinyl)anthracene-9,10-dione trifluoroacetate salt [Method 6]

Prepared by deprotection of example (14). ¹H nmr spectrum (d₆-DMSO)(200 MHz) δ: 2.7 (2H, m); 3.2 (4H, m); 3.7 (4H, m); 3.15 (2H, m); 3.7 (4H, m); 4.5 (2H, t), 7.4 (1H, dd); 7.6-8.2 (9H, m, unresolved). ESMS(+) m/z: 364 (100%)(RNH₃)⁺. ESMS(−) m/z: 113 (100%). M, 477.

### EXAMPLE (16)

### 1-(1-N-tertiarybutoxycarbonyl-L-alanylpiperazinyl)anthracene-9,10-dione.[Method 4]

Prepared from anthraquinone-spacer compound (4a) and N-tertiarybutoxycarbonyl-L-alanine N-hydroxysuccinimide ester. Mp 110° C. ¹H nmr spectrum (CDCl₃)(200 MHz) δ: 1.35 (3H, d); 1.45 (9H, s); 3.10-3.30 (4H, m); 3.80-4.20 (4H, m); 4.70 (1H, m); 5.60 (1H, d); 7.25 (1H, s); 7.65-7.85 (2H, m); 7.75 (3H, m); 8.00 (1H, d); 8.20-8.30 (2H, m). FABMS(+) m/z 486 (3%), 464 (100%)(MH)⁺. M, 463.

### EXAMPLE (17)

### 1-(1-L-alanylpiperazinyl)anthracene-9,10-dione trifluoroacetate salt. [Method 6]

Prepared by deprotection of example (16). ¹H nmr (d₆-DMSO)(200 MHz) δ: 1.35 (3H, d); 3.2 (4H, m); 3.8 (4H, m); 4.5 (1H, q); 7.55 (1H, dd); 7.70-7.80 (4H, m); 8.10-8.50 (5H, m). ESMS(+) m/z: 364 (100%)(RNH₃)⁺. ESMS(−) m/z: 113 (55%), 69 (100%). M, 477.

### EXAMPLE (18)

### 1-(1-N-tertiarybutoxycarbonyl-L-valylpiperazinyl)anthracene-9,10-dione. [Method 4]

Prepared from anthraquinone-spacer compound (4a) and N-tertiarybutoxycarbonyl-L-valine N-hydroxysuccinimide ester. ¹H nmr spectrum (CDCl₃)(200 MHz) δ:1.10 (6H, m); 1.45 (9H, s); 2.30 (1H, m); 3.20 (4H, m); 3.90 (4H, m); 4.55 (1H, dd); 5.40 (1H, d); 7.35 (1H, dd); 7.60-7.85 (3H, m); 8.05 (1H, dd); 8.20-8.30 (2H, m). FABMS(+) m/z: 514 (5%), 492 (100%)(MH)⁺. M, 491.

### EXAMPLE (19)

### 1-(1-L-valylpiperazinyl)anthracene-9,10-dione trifluoroacetate salt. [Method 6]

Prepared by deprotection of example (18). ¹H nmr (d₆-DMSO)(200 MHz) δ: 0.80-1.10 (6H, m); 2.10 (1H, m); 3.2 (4H, m); 3.70-3.40 (4H, m); 4.40 (1H, br.s); 7.55 (1H, dd); 7.70-7.80 (4H, m); 8.00-8.30 (5H, m). ESMS(+) m/z: 392 (100%)(RNH₃)⁺. ESMS(−) m/z: 113 (100%). M, 505.

### EXAMPLE (20)

### 1-(1-N-tertiarybutoxycarbonyl-L-phenylalanylpiperazinyl)anthracene-9,10-dione. [Method 4]

Prepared from anthraquinone-spacer compound (4a) and N-tertiarybutoxycarbonyl-L-phenylalanine N-hydroxysuccinimide ester. ¹H nmr spectrum (CDCl₃)(200 MHz) δ: 1.45 (9H, s); 2.85-3.30 (6H, m); 3.60-3.95 (4H, m); 4.90 (1H, qn); 5.60 (1H, d); 7.15-7.35 (6H, m); 7.60-7.85 (3H, m); 8.00 (1H, dd); 8.20-8.30 (2H, m). FABMS(+) m/z 562 (8%), 540 (100%)(MH)⁺. M, 539.

### EXAMPLE (21)

### 1-(1-L-phenylalanylpiperazinyl)anthracene-9,10-dione trifluoroacetate salt. [Method 6]

Prepared by deprotection of example (20). ¹H nmr (d₆-DMSO)(200 MHz) δ: 2.85-3.20 (6H, m); 3.30 (2H, m); 3.65 (2H, m); 4.70 (1H, mt); 7.15-7.45 (7H, m); 7.70-7.95 (3H, m); 8.05-8.20 (2H, m); 8.30 (3H, br.s). ESMS(+) m/z: 462 (3%), 440 (100%)(RNH₃)⁺. ESMS(−) m/z: 113 (60%), 69 (100%). M, 553.

### EXAMPLE (22)

### 1,5-bis(1-N-tertiarybutoxycarbonyl-L-alanylpiperazinyl)anthracene-9,10-dione.[Method 4]

Prepared from anthraquinone-spacer compound (8) and N-tertiarybutoxycarbonyl-L-alanine N-hydroxysuccinimide ester (2.2 eq). Mp 156° C. FABMS(+) m/z: 741 (25%)(MNa)⁺, 719 (100%)(MH)⁺. M, 720.

### EXAMPLE (23)

### 1,5-bis(1-L-alanylpiperazinyl)anthracene-9,10-dione trifluoroacetate salt. [Method 6]

Prepared by deprotection of example (22). Mp 182° C. ESMS(+) m/z: 541 (2%), 519 (80%)[(RNH)₂NH₃]⁺, 260 (100%)(RNH₃)²⁺. ESMS(−) m/z: 113 (55%), 69 (100%). M, 746.

### EXAMPLE (24)

### 1-[(4-aminocyclohexyl)amino])anthracene-9,10-dione. [Method 3]

Prepared using trans-1,4-diaminocyclohexane and 1-chloroanthraquinone. CIMS(+) m/z 321 (10%)(MH)⁺, 260 (100%), 243 (80%). M, 320.

### EXAMPLE (25)

### (2S)-2-[(tert-butoxy)carbonylamino]-N-{4-[(9,10-dioxoanthryl)amino]-cyclohexyl]-3-phenylpropanamide. [Method 4]

Prepared from anthraquinone-spacer compound (24) and N-tertiarybutoxycarbonyl-L-phenylalanine N-hydroxysuccinimide ester. Mp 144° C. FABMS(+) m/z: 568 (8%)(MH)⁺, 385 (45%), 120 (100%). M, 567.

### EXAMPLE (26)

### (2S)-2-amino-N-{4-[(9,10-dioxoanthryl)amino]cyclohexyl}-3-phenylpropanamide trifluoroacetate salt. [Method 6]

Prepared by deprotection of example (25). Mp 87° C. ESMS(+)(Cone 8V) m/z: 468 (100%)(RNH₃)⁺. ESMS(−) m/z: 113 (100%). M, 581.

### EXAMPLE (27)

### (2R)-2-[(tert-butoxy)carbonylamino]-N-{4-[(9,10-dioxoanthryl)amino]-cyclohexyl}-3-phenylpropanamide. [Method 4]

Prepared from anthraquinone-spacer compound (24) and N-tertiarybutoxycarbonyl-D-phenylalanine N-hydroxysuccinimide ester. Mp 142° C. FABMS(+) m/z: 568 (8%)(MH)⁺, 385 (45%), 120 (100%). M, 567.

### EXAMPLE (28)

### (2R)-2-amino-N-{4-[(9,10-dioxoanthryl)amino]cyclohexyl}-3-phenylpropanamide trifluoroacetate salt. [Method 6]

Prepared by deprotection of example (27). Mp 86° C. ESMS(+) m/z 935 (2%) (2M−H), 468 (100%)(RNH₃)⁺. M, 581.

### EXAMPLE (29)

### 2-[(tert-butoxy)carbonylamino]-N-{4-[9,10-dioxoanthryl)amino]-cyclohexyl}acetamide.[Method 4]

Prepared from anthraquinone-spacer compound (24) and N-tertiarybutoxycarbonyl-glycine N-hydroxysuccinimide ester. CIMS(+) m/z: 478 (100%)(MH)⁺.M, 477

### EXAMPLE (30)

### 2-amino-N-{4-[(9,10-dioxoanthryl)amino]cyclohexyl}acetamide trifluoroacetate salt. [Method 6]

Prepared by deprotection of example (29). Mp 229° C. ¹H nmr spectrum (d₆-DMSO)(200 MHz) δ: 1.65 (4H, m); 1.85 (2H, m); 2.15 (2H, m); 3.50 (2H, s); 3.70 (1H, m); 7.40 (2H, m); 7.65 (1H, m); 7.70-8.10 (5H, m); 8.20 (2H, m); 9.75 (1H, d). FABMS (+) m/z: 400 (10%)(RNH₃+Na)⁺, 378 (100%)(RNH₃)⁺. M, 491.

### EXAMPLE (31)

### (2S)-2-[(tert-butoxy)carbonylamino]-N-{4-[(9,10-dioxoanthryl)amino]-cyclohexyl}propanamide. [Method 4]

Prepared from anthraquinone-spacer compound (24) and N-tertiarybutoxycarbonyl-L-alanine N-hydroxysuccinimide ester. Mp 151° C. CIMS(+) m/z: 492 (10%)(MH)⁺, 417 (100%), 392 (12%). M, 491.

### EXAMPLE (32)

### (2S)-2-amino-N-{4-[(9,10-dioxoanthryl)amino]cyclohexyl}propanamide trifluoroacetate salt. [Method 6]

Prepared by deprotection of example (31). Mp 120° C. ¹H nmr spectrum (d₆-DMSO)(200 MHz) δ: 1.20 (3H, t,); 1.80 (4H, br.s); 2.20 (4H, br.s); 3.20 (1H, s); 7.30-7.40 (2H, unresolved, m); 7.60 (1H, t); 7.70-7.90 (2H, m); 8.00-8.30 (2H, m); 8.40 (1H, d); 9.75 (1H, d). ESMS(+) m/z: 392 (100%)(RNH₃)⁺. M, 505.

### EXAMPLE (33)

### (2R)-2-[(tert-butoxy)carbonylamino]-N-{4-[(9,10-dioxoanthryl)amino]-cyclohexyl}propanamide. [Method 4]

Prepared from anthraquinone-spacer compound (24) and N-tertiarybutoxycarbonyl-D-alanine N-hydroxysuccinimide ester. Mp 152° C. CIMS(+) m/z: 492 (15%)(MH)⁺, 417 (100%), 391 (50%). M, 491.

### EXAMPLE (34)

### (2R)-2-amino-N-{4-[(9,10-dioxoanthryl)amino]cyclohexyl}propanamide trifluoroacetate salt. [Method 6]

Prepared by deprotection of example (33). Mp 118° C. ESMS(+) m/z: 414 (17%), 392 (100%)(RNH₃)⁺. ESMS(−) m/z: 113 (90%), 69 (100%). M, 505.

### EXAMPLE (35)

### (2S)-2-[(tert-butoxy)carbonylamino]-N-{4-[(9,10-dioxoanthryl)amino]-cyclohexyl}-3-methylbutanamide. [Method 4]

Prepared from anthraquinone-spacer compound (24) and N-tertiarybutoxycarbonyl-L-valine N-hydroxysuccinimide ester. Mp 179° C. EIMS(+) m/z: 519 (5%)(MH)⁺, 419 (4%), 49 (100%). M, 519.

### EXAMPLE (36)

### (2S)-2-amino-N-{4-[(9,10-dioxoanthryl)amino]cyclohexyl}-3-methylbutanamide trifluoroacetate salt. [Method 6]

Prepared by deprotection of example (35). Mp 182° C. ESMS(+)(Cone8V) m/z: 420 (100%)(RNH₃)⁺. ESMS(−)(Cone 8V) m/z: 113 (35%), 69 (100%). M, 533.

### EXAMPLE (37)

### (2R)-2-[(tert-butoxy)carbonylamino]-N-{4-[(9,10-dioxoanthryl)amino]-cyclohexyl}-3-methylbutanamide. [Method 4]

Prepared from anthraquinone-spacer compound (24) and N-tertiarybutoxycarbonyl-D-valine N-hydroxysuccinimide ester. Mp 181° C. EIMS(+) m/z: 519 (3%)(MH)⁺, 419 (3%), 49 (100%). M, 519.

### EXAMPLE (38)

### (2R)-2-amino-N-{4-[(9,10-dioxoanthryl)amino]cyclohexyl}-3-methylbutanamide trifluoroacetate salt. [Method 6]

Prepared by deprotection of example (37). Mp 178° C. ESMS(+) m/z: 442 (10%), 420 (42%)(RNH₃)⁺, 197 (100%). ESMS(−) m/z: 113 (80%), 69 (100%). M, 533.

### EXAMPLE (39)

### tert-butyl (2S)-2-(N-{4-[(9,10-dioxoanthryl)amino]-cyclohexyl}carbonyl)pyrrolidine carboxylate. [Method 4]

Prepared from anthraquinone-spacer compound (24) and N-tertiarybutoxycarbonyl-L-proline N-hydroxysuccinimide ester. Mp 157° C. FABMS (+) m/z: 540 (35%)(M+Na)⁺, 518 (100%)(MH)⁺. 419 (12%), 195 (40%), 121 (95%). M, 517.

### EXAMPLE (40)

### ((2S)pyrrolidin-2-yl)-N-{4-[(9,10-dioxoanthryl)amino]cyclohexyl}-carboxamide trifluoroacetate salt. [Method 6]

Prepared by deprotection of example (39). Mp 210° C. FABMS (+) m/z: 440 (12%)(RNH₃+Na)⁺, 418 (100%)(RNH₃)⁺. M, 531.

### EXAMPLE (41)

### (2S)-2-[(tert-butoxy)carbonylamino]-N-{4-[(9,10-dioxoanthryl)amino]-cyclohexyl}-3-(4-hydroxyphenyl)propanamide. [Method 4]

Prepared from anthraquinone-spacer compound (24) and N-tertiarybutoxycarbonyl-L-tyrosine N-hydroxysuccinimide ester. FABMS(+) m/z: 606 (3%), 584 (100%)(MH)⁺. M, 583.

### EXAMPLE (42)

### (2S)-2-amino-N-{4-[(9,10-dioxoanthryl)amino]cyclohexyl}-3-(4-hydroxyphenyl)propanamide trifluoroacetate salt. [Method 6]

Prepared by deprotection of example (41). Mp 198° C. FABMS (+) m/z: 506 (16%)(RNH₃+Na)⁺, 484 (65%)(RNH₃)⁺. 345 (30%), 182 (100%). M, 597.

### EXAMPLE (43)

### (2S)-2-[(tert-butoxy)carbonylamino]-N-{4-[(9,10-dioxoanthryl)amino]-cyclohexyl}-3-(2-chlorophenyl)propanamide. [Method 4]

Prepared from anthraquinone-spacer compound (24) and N-tertiarybutoxycarbonyl-L-(2-chloro)phenylalanine N-hydroxysuccinimide ester. Mp 185° C. FABMS (+) m/z: 624 (20%)(M+Na)⁺, 602 (35%)(MH)⁺. 546 (40%), 279 (26%), 225 (100%). M, 602.

### EXAMPLE (44)

### (2S)-2-amino-N-{4-[(9,10-dioxoanthryl)amino]cyclohexyl}-3-(2-chlorophenyl)propanamide trifluoroacetate salt. [Method 6]

Prepared by deprotection of example (43). Mp 202° C. ESMS(+)(Cone 20V) m/z: 534 (2%), 502 (100%)(RNH₃)⁺, 281 (30%), 132 (75%). ESMS(−)(Cone 20V) m/z: 113 (100%). M, 615.

### EXAMPLE (45)

### (2R)-2-[(tert-butoxy)carbonylamino]-N-{4-[(9,10-dioxoanthryl)amino]-cyclohexyl}-3-(2-chlorophenyl)propanamide. [Method 4]

Prepared from anthraquinone-spacer compound (24) and N-tertiarybutoxycarbonyl-D-(2-chloro)phenylalanine N-hydroxysuccinimide ester. Mp 183° C. FABMS(+) m/z: 624 (46%), 602 (100%)(MH)⁺. M, 602.

### EXAMPLE (46)

### (2R)-2-amino-N-{4-[(9,10-dioxoanthryl)amino]cyclohexyl}-3-(2-chloro-phenyl)propanamide trifluoroacetate salt. [Method 6]

Prepared by deprotection of example (45). Mp 206° C. ESMS(+)(Cone 20V) m/z: 502 (100%)(RNH₃)⁺. ESMS(−)(Cone 20V) m/z: 113 (100%). M, 615.

### EXAMPLE (47)

### 1-({3-[4-(3-aminopropyl)piperazinyl]propyl}amino)anthracene-9,10-dione [Method 3]

Prepared using 1,4-bis(3-aminopropyl)piperazine and 1-Chloroanthraquinone. FABMS(+) m/z: 407 (30%)(MH)⁺, 201 (100%). M, 406.

### EXAMPLE (48)

### 2-[(tert-butoxy)carbonylamino]-N-[3-(4-{3-[(9,10-dioxoanthryl)amino]-propyl}piperazinyl)propyl]acetamide. [Method 4]

Prepared from anthraquinone-spacer compound (47) and N-tertiarybutoxycarbonyl-glycine N-hydroxysuccinimide ester. Mp 118° C. CIMS(+) m/z: 564 (100%)(MH)⁺. M, 563.

### EXAMPLE (49)

### 2-amino-N-[3-(4-{3-[(9,10-dioxoanthryl)amino]propyl}piperazinyl)-propyl]acetamide tri-trifluoroacetate salt. [Method 6]

Prepared by deprotection of example (48). FABMS(+) m/z: 464 (29%)(RNH₃)⁺, 23 (100%).

### EXAMPLE (50)

### (2S)-2-[(tert-butoxy)carbonylamino]-N-[3-(4-{3-[(9,10-dioxoanthryl)-amino]propyl}piperazinyl)propyl]propanamide. [Method 4]

Prepared from anthraquinone-spacer compound (47) and N-tertiarybutoxycarbonyl-L-alanine N-hydroxysuccinimide ester. CIMS(+) m/z: 578 (45%)(MH)⁺, 504 (95%), 210 (100%). M, 577.

### EXAMPLE (51)

### (2S)-2-amino-N-[3-(4-{3-[(9,10-dioxoanthryl)amino]propyl}-piperazinyl)propyl]propanamide trifluoroacetate salt. [Method 6]

Prepared by deprotection of example (50). FABMS(+) m/z: 500 (6%), 478 (100%)(RNH₃)⁺, 154 (80%), 44 (95%).

### EXAMPLE (52)

### (2R)-2-[(tert-butoxy)carbonylamino]-N-[3-(4-{3-[(9,10-dioxoanthryl)amino]propyl}piperazinyl)propyl]propanamide. [Method 4]

Prepared from anthraquinone-spacer compound (47) and N-tertiarybutoxycarbonyl-D-alanine N-hydroxysuccinimide ester. Mp 78-80° C. FABMS(+) m/z: 578 (8%)(MH)⁺, 385 (45%), 120 (100%). M, 577.

### EXAMPLE (53)

### (2R)-2-amino-N-[3-(4-{3-[(9,10-dioxoanthryl)amino]propyl}-piperazinyl)propyl]propanamide trifluoroacetate salt. [Method 6]

Prepared by deprotection of example (52). Mp 120° C. ESMS(+)(Cone 20V) m/z: 478 (30%)(RNH₃)⁺, 239 (100%). ESMS(−)(Cone 20V) m/z: 113 (100%).

### EXAMPLE (54)

### (2S)-2-[(tert-butoxy)carbonylamino]-N-[3-(4-{3-[(9,10-dioxoanthryl)-amino]propyl}piperazinyl)propyl]-3-methylbutanamide. [Method 4]

Prepared from anthraquinone-spacer compound (47) and N-tertiarybutoxycarbonyl-L-valine N-hydroxysuccinimide ester. Mp 108° C. FABMS(+) m/z: 628 (5%)(M+Na)⁺, 606 (100%)(MH)⁺. M, 605.

### EXAMPLE (55)

### (2S)-2-amino-N-[3-(4-{3-[(9,10-dioxoanthryl)amino]propyl}-piperazinyl)propyl]-3-methylbutanamide trifluoroacetate salt. [Method 6]

Prepared by deprotection of example (54). Mp 79° C. FABMS(+) m/z: 528 (10%)(RNH₃+Na)⁺, 506 (85%)(RNH₃)⁺, 399 (100%).

### EXAMPLE (56)

### tert-butyl (2S)-2-{N-[3-(4-{3-[(9,10-dioxoanthryl)amino]propyl}-piperazinyl)propyl]carbomyl}pyrrolidinecarboxylamide. [Method 4]

Prepared from anthraquinone-spacer compound (47) and N-tertiarybutoxycarbonyl-L-proline N-hydroxysuccinimide ester. Mp 72° C. CIMS(+) m/z: 604 (5%)(MH)⁺, 504 (8%), 70 (100%). M, 603.

### EXAMPLE (57)

### ((2S)pyrrolidine-2-yl)-N-[3-(4-{3-[(9,10-dioxoanthryl)amino]propyl}-piperazinyl)propyl]carboxamide trifluoroacetate salt. [Method 6]

Prepared by deprotection of example (56). ESMS(+)(Cone 50V) m/z: 505 (90%)(RNH₃)⁺, 96 (100%) ESMS(−)(Cone 50V) m/z: 113 (100%).

### EXAMPLE (58)

### (2S)-2-[(tert-butoxy)carbonylamino]-N-[3-(4-{3-[(9,10-dioxoanthryl)-amino]propyl}piperazinyl)propyl]-3-phenylpropanamide. [Method 4]

Prepared from anthraquinone-spacer compound (47) and N-tertiarybutoxycarbonyl-L-phenylalanine N-hydroxysuccinimide ester. Mp 168° C. ¹H nmr spectrum (CDCl₃)(200 MHz) δ: 1.40 (9H, s); 1.55 (2H, m); 1.90 (2H, m); 2.20-2.55 (10H, m); 3.00 (2H, m); 3.20-3.45 (4H,m); 4.20 (1H, m); 5.15 (1H, d); 7.00 (1H, br. s); 7.10 (1H, dd); 7.20 (5H, m); 7.50 (2H, m); 8.70 (2H, m); 8.20 (2H, m); 9.80 (1H, t). FABMS(+) m/z: 676 (12%)(M+Na)⁺, 654 (100%)(MH)⁺, 236 (16%). M, 653.

### EXAMPLE (59)

### (2S)-2-amino-N-[3-(4-{3-[(9,10-dioxoanthryl)amino]propyl}-piperazinyl)propyl]-3-phenylpropanamide trifluoroacetate salt [Method 6]

Prepared by deprotection of example (58). ESMS(+)m/z: 554 (100%)(RNH₃)⁺. ESMS(−)(Cone50 V) m/z: 113 (100%).

### EXAMPLE (60)

### (2R)-2-[(tert-butoxy)carbonylamino]-N-[3-(4-{3-[(9,10-dioxoanthryl)-amino]propyl}piperazinyl)propyl]-3-phenylpropanamide [Method 4]

Prepared from anthraquinone-spacer compound (47) and N-tertiarybutoxycarbonyl-D-phenylalanine N-hydroxysuccinimide ester. CIMS(+) m/z: 676 (10%)(M+Na)⁺, 654 (100%)(MH)⁺, 236 (11%). M, 653.

### EXAMPLE (61)

### (2R)-2-amino-N-[3-(4-{3-[(9,10-dioxoanthryl)amino]propyl}-piperazinyl)propyl]-3-phenylpropanamide trifluoroacetate salt [Method 6]

Prepared by deprotection of example (60). ESMS(+)(Cone50V) m/z: 577 (6%), 554(100%)(RNH₃)⁺. ESMS(−)(Cone50 V) m/z: 113(100%).

### EXAMPLE (62)

### (2S)-2-[(tert-butoxy)carbonylamino]-N-[3-(4-{3-[(9,10-dioxoanthryl)-amino]propyl}piperazinyl)propyl]-3-(4-hydroxyphenyl)propanamide [Method 4]

Prepared from anthraquinone-spacer compound (47) and N-tertiarybutoxycarbonyl-L-phenylalanine N-hydroxysuccinimide ester. Mp 102° C. FABMS (+) m/z: 692 (3%)(M+Na)⁺, 670 (100%)(MH)⁺. 419 (10%), 236 (15%), 121 (43%). M, 669.

### EXAMPLE (63)

### (2S)-2-amino-N-[3-(4-{3-[(9,10-dioxoanthryl)amino]propyl}-piperazinyl)propyl]-3-(4-hydroxyphenyl)propanamide trifluoroacetate salt. [Method 6]

Prepared by deprotection of example (62). Mp 148° C. FABMS (+) m/z: 592 (8%)(RNH₃+Na)⁺, 570 (100%)(RNH₃)⁺. 391 (12%), 260 (20%).

### EXAMPLE (64)

### 1-[(2-aminocyclohexyl)amino]anthracene-9,10-dione. [Method 3]

Prepared using trans-1,2-diaminocyclohexane and 1-Chloroanthraquinone. FABMS(+) m/z: 343 (2%), 321 (100%)(MH)⁺. M, 320.

### EXAMPLE (65)

### (2S)-2-[(tert-butoxy)carbonylamino]-N-{2-[(9, 1-dioxoanthryl)amino]-cyclohexyl}propanamide. [Method 4]

Prepared from anthraquinone-spacer compound (64) and N-tertiarybutoxycarbonyl-L-alanine N-hydroxysuccinimide ester. FABMS(+) m/z: 514 (8%)(M+Na)⁺, 492, (100%)(MH)⁺. M, 491.

### EXAMPLE (66)

### (2S)-2-amino-N-{2-[(9,10-dioxoanthryl)amino]cyclohexyl}propanamide trifluoroacetate salt. [Method 6]

Prepared by deprotection of example (65). ¹H nmr spectrum (d₆-DMSO)(200 MHz) δ: 1.20-1.60 (7H, m, unresolved); 1.60-2.25 (4H, m, unresolved); 3.60 (1H, m); 3.85 (1H, m); 4.10 (1H, m); 7.40-7.55 (2H, m, unresolved); 7.65 (1H, m); 7.80-7.95 (2H, m); 8.00-8.25 (5H, m, unresolved); 8.45 (1H, m); 9.85 (1H, m). FABMS(+) m/z: 414 (4%), 392 (100%)(RNH₃)⁺. M, 505.

### EXAMPLE (67)

### N-[2-(N-{2-[(9,10-dioxoanthryl)amino]cyclohexyl}carbamoyl)ethyl]-2,2-dimethylpropanamide. [Method 4]

Prepared from anthraquinone-spacer compound (64) and N-tertiarybutoxycarbonyl-β-alanine N-hydroxysuccinimide ester. Mp 168° C. CIMS(+) m/z: 492 (20%)(MH)⁺, 98 (100%). M, 491.

### EXAMPLE (68)

### 3-amino-N-{2-[(9,10-dioxoanthryl)amino]cyclohexyl}propanamide trifluoroacetate salt. [Method 6]

Prepared by deprotection of example (67). Mp 238° C. FABMS(+) m/z: 414 (10%)(MNa)⁺, 392 (100%)(RNH₃)⁺. M, 505.

### EXAMPLE (69)

### 2-[(tert-butoxy)carbonylamino]-N-{4-[(9,10-dioxoanthryl)amino]-phenyl}acetamide. [Method 4]

Prepared from anthraquinone-spacer compound (71) and N-tertiarybutoxycarbonyl-glycine N-hydroxysuccinimide ester. Mp 171° C. EIMS(+) m/z: 471 (20%)(M)⁺, 397 (100%), 371 (40%). M, 471.

### EXAMPLE (70)

### 2-amino-N-{4-[(9,10-dioxoanthryl)amino]phenyl}acetamide trifluoroacetate salt. [Method 6]

Prepared by deprotection of example (69). Mp 221° C. ESMS(+)(Cone20V) m/z: 372 (100%)(RNH₃)⁺. ESMS(−)(Cone20V) m/z: 113 (100%). M, 485.

### EXAMPLE (71)

### 1-[(4-aminophenyl)amino]anthracene-9,10-dione. [Method 3]

1-Chloroanthraquinone (3 mmol) was suspended in DMSO (10 cm³); 1,4-phenylenediamine (20 mmol) was added and the mixture was heated at reflux for 1 h The solution was cooled, methanol was added (50 cm³) and mixture stirred at 0° C. Di-tertiarybutyl-dicarbonate (10 mmol) in methanol (20 cm³) was added dropwise and the reaction mixture was allowed to reach room temperature. The crude N-^{t}Boc protected compound was extracted into chloroform before purification by silica gel chromatography [chloroform:ethyl acetate (4:1)] to give the N-^{t}Boc protected spacer compound which was deprotected using trifluoroacetic acid [Method 6] and neutralised using triethylamine to give the title compound. Mp 175-182° C. FABMS(+) m/z: 315 (100%)(MH)⁺. M, 314.

### EXAMPLE (72)

### 1-[(2-piperazinylethyl)amino]anthracene-9,10-dione. [Method 3]

Prepared using 2-piperazinylethylamine and 1-Chloroanthraquinone. CIMS(+) m/z: 336 (30%)(MH)⁺, 130 (100%). M, 335.

### EXAMPLE (73)

### N-[(1S)-2-(4-{2-[(9,10-dioxoanthryl)amino]ethyl}piperazinyl)-1-methyl-2-oxoethyl](tert-butoxy)carboxamide. [Method 4]

Prepared from anthraquinone-spacer compound (72) and N-tertiarybutoxycarbonyl-L-alanine N-hydroxysuccinimide ester. ¹H nmr spectrum (CDCl₃)(200 MHz) δ: 1.35 (3H, d); 1.45 (9H, s); 2.50-2.65 (4H, m); 2.80 (2H, t); 3.45 (2H, m); 3.50-3.90 (4H, m); 4.60 (1H, m); 5.55 (1H, d); 7.05 (1H, dd); 7.50-7.60 (2H, m); 7.65-7.80 (2H, m); 8.20-8.30 (2H, m); 9.75 (1H, t).

### EXAMPLE (74)

### 1-({2-[4-((2S)-2-aminopropanoyl)piperazinyl]ethyl}amino]anthracene-9,10-dione trifluoroacetate salt. [Method 6]

Prepared by deprotection of example (73). FABMS(+) m/z: 430 (5%)(MNa)⁺, 408 (100%)(RNH₃)⁺.

### EXAMPLE (75)

### 1-[(4-piperidylmethyl)amino]anthracene-9,10-dione. [Method 3]

Prepared using 4-(aminomethyl)piperidine and 1-Chloroanthraquinone. FABMS(+) m/z: 321 (100%)(RNH₃)⁺. M, 320.

### EXAMPLE (76)

### N-[(1S)-2-(4-{[(9,10-dioxoanthryl)amino]methyl}piperidyl)-2oxo-1-benzylethyl](tert-butoxy)carboxamide. [Method 4]

Prepared from anthraquinone-spacer compound (75) and N-tertiarybutoxycarbonyl-L-phenylalanine N-hydroxysuccinimide ester. Mp 178° C. FABMS(+) m/z: 590 (15%)(M+Na)⁺, 568 (100%)(MH)⁺, 512 (18%). M, 567.

### EXAMPLE (77)

### 1-({[1-((2S)-2-amino-3-phenylpropanoyl)4-piperidyl]methyl}amino]-anthracene-9,10-dione trifluoroacetate salt. [Method 6]

Prepared by deprotection of example (76). ESMS(+)(Cone20V) m/z: 478 (100%)(RNH₃)⁺. ESMS(−)(Cone20V) m/z: 113 (35%), 69 (100%). M, 581.

### EXAMPLE (78)

### N-{(5S)-5-[(tertiarybutoxycarbonylamino]-6-[4-(9,10-dioxoanthryl)-piperazinyl)-6-oxohexyl}(phenylmethoxy)carboxamide.[Method 4]

Prepared from anthraquinone-spacer compound (4a) and N-α-tertiarybutoxycarbonyl-N-ε-benzyloxycarbonyl-L-lysine N-hydroxysuccinimide ester. Mp 90° C. FABMS(+) m/z: 677 (27%)(M+Na)⁺, 655 (100%)(MH)⁺, 599 (10%), 555 (5%). M, 654.

### EXAMPLE (79)

### 1-[4-((2S)-2-6-diaminohexanoyl)piperazinyl]anthracene-9,10-dione bis trifluoroacetate salt [Method 6]

Prepared by deprotection of example (78). Mp 108° C. ESMS(+)(Cone50V) m/z: 421 (40%)[(RNH)₂NH₃]⁺, 293 (100%). ESMS(+)(Cone20V) m/z: 113 (45%), 69 (100%). M, 648.

### EXAMPLE (80)

### 1-[(4-aminocyclohexyl)amino]-4-hydroxyanthracene-9,10-dione trifluoroacetate salt

1,4-Dihydroxyanthraquinone (2 mmol) was suspended in ethanol (25 cm³) and THF (25 cm³) containing trans-1,4-diaminocyclohexane (20 mmol) and heated over a water bath (at 95° C.) for 1.75 h. The solution was cooled and di-tertiarybutyldicarbonate (8 mmol) in methanol (20 cm³) was added dropwise and the reaction mixture was allowed to reach room temperature. The crude N-^{t}Boc protected compound was extracted into chloroform and applied to a silica gel chromatography column, using toluene:ethyl acetate (4:1) as the eluting solvent, to give the N-⁴Boc protected compound ( ) as a purple solid after recrystallisation of the residue from the major fraction from ethanol. The N-^{t}Boc protected compound was deprotected using trifluoroacetic acid [Method 6] to give the title compound. FABMS(+) m/z: 451 (100%)(MH)⁺. M, 450.

### EXAMPLE (81)

### 4-[(4-aminocyclohexyl)amino]-1,5-dihydroxyanthracene-9,10-dione trifluoroacetate salt

Leuco-1,4,5-trihydroxyanthraquinone (1 mmol) was suspended in dichloromethane (50 cm³). Trans-1,4-diaminocyclohexane (1 mmol) was added and the mixture was stirred at room temperature for 6 h followed by the addition of triethylamine (2 cm³) and aeration for 2 h. The solvent was evaporated and the mixture suspended in methanol and stirred at 0° C. Di-tertiarybutyl-dicarbonate (3 mmol) in methanol (20 cm³) was added dropwise and the reaction mixture was allowed to reach room temperature. The crude N-^{t}Boc protected compound was extracted into chloroform before applying to a silica gel chromatography column (4×40 cm) prepared with chloroform. The column was eluted firstly with chloroform before the addition of ethyl acetate to give the N-^{t}Boc protected spacer compound which was deprotected using trifluoroacetic acid [Method 6] to give the title compound. FABMS(+) m/z: 467 (100%)(MH)⁺. M, 466.

### EXAMPLE (82)

### (2S)-2-[(tert-butoxy)carbonylamino]-N-{4-[(4-hydroxy-9,10-dioxoanthryl)amino]cyclohexyl}-3-phenylpropanamide. [Method 4]

Prepared by the reaction of anthraquinone-spacer compound (80) and N-tertiarybutoxycarbonyl-L-phenylalanine N-hydroxysuccinimide ester in THF and triethylamine (1 eq). FABMS(+) m/z: 606 (5%), 584 (100%)(MH)⁺. M, 583.

### EXAMPLE (83)

### (2S)-2-amino-N-{4-[(4-hydroxy-9,10-dioxoanthryl)amino]-cyclohexyl}-3-phenylpropanamide trifluoroacetate salt. [Method 6]

Prepared by deprotection of example (82). ESMS(+)(Cone20V) m/z: 484 (100%)(RNH₃)⁺. ESMS(−)(Cone20V) m/z: 113 (100%). M, 597.

### EXAMPLE (84)

### (2S)-N-2-[(tert-butoxy)carbonylamino]-N-{4-[(4-hydroxy-9,10-dioxoanthryl)amino]cyclohexyl}-3-phenylpropanamide. [Method 4]

Prepared by the reaction of anthraquinone-spacer compound (81) and N-tertiarybutoxycarbonyl-L-phenylalanine N-hydroxysuccinimide ester in THF and triethylamine (1 eq). CIMS(+) m/z: 600 (100%)(MH)⁺. M, 599. C₃₄H₃₇N₃O₇ requires C, 68.1; H, 6.2; N, 7.0%. Found C, 68.4; H, 6.0; N, 6.8%.

### EXAMPLE (85)

### 1-[4-(3-(4-piperidyl)propyl)piperidyl]anthracene-9,10-dione. [Method 1]

Prepared using 4,4′-trimethylenedipiperidine and 1-Chloroanthraquinone. FABMS(+) m/z: 439 (8%), 417 (100%)(MH)⁺. M, 416.

### EXAMPLE (86)

### N-[(1S)-2-(4-{3-[1-(9,10-dioxoanthryl)(4-piperidyl)]propyl}piperidyl)-2-oxo-1-propylethyl}(tert-butoxy)carboxamide. [Method 4]

Prepared from anthraquinone-spacer compound (85) and N-tertiarybutoxycarbonyl-L-norvaline pentafluorophenolate ester. CIMS(+) m/z: 616 (100%)(MH)⁺. M, 615. C₃₇H₄₉N₃O₅ requires C, 72.2; H, 8.0; N, 6.8%. Found C, 71.8; H, 8.1; N, 6.6%.

### EXAMPLE (87)

### 1-(4-{3-[1-((2S)-2-aminopentanoyl)-4-piperidyl]propyl}piperidyl)-anthracene-9,10-dione trifluoroacetate salt. [Method 6]

Prepared by deprotection of example (86). ESMS(+)(Cone20V) m/z: 516 (100%)(RNH₃)⁺. ESMS(−)(Cone20V) m/z; 113 (45%), 69 (100%). M, 629.

### EXAMPLE (88)

### N-[1-((1R)-1-methylpropyl)(1S)-2-(4-{3-[1-(9,10-dioxoanthryl)(4-piperidyl)]propyl}piperidyl)-2-oxoethyl](tert-butoxy)carboxamide. [Method 4]

Prepared from anthraquinone-spacer compound (85) and N-tertiarybutoxycarbonyl-L-isoleucine N-hydroxysuccinimide ester. CIMS(+) m/z: 630 (100%)(MH)⁺. M, 629.

### EXAMPLE (89)

### 1-(4-{3-[1-((2S,3R)-2-amino-3-methylpentanoyl)-4-piperidyl]propyl}-piperidyl)anthracene-9,10-dione trifluoroacetate salt. [Method 6]

Prepared by deprotection of example (88). FABMS(+) m/z: 552 (5%), 530 (100%)(RNH₃)⁺. M, 643.

### EXAMPLE (90)

### 1,5-bis[(4-aminophenyl)amino]anthracene-9,10-dione. [Method 2]

Prepared by the reaction of N-(4-aminophenyl)(tert-butoxy)carboxamide (5 eq) and 1,5-dichloroanthraquinone (1 eq). The crude N-^{t}Boc protected compound was extracted into chloroform before purification by silica gel chromatography [chloroform:ethyl acetate (4:1)] to give the N-^{t}Boc protected spacer compound which was deprotected using trifluoroacetic acid [Method 6] and neutralised using triethylamine to give the title compound. FABMS(+) m/z: 421 (100%)(MH)⁺. M, 420.

### EXAMPLE (91)

### 2-[(tert-butoxy)carbonylamino]-N-[4-({5-[(4-{2-[(tert-butoxy)carbonyl-amino]acetylamino}phenyl)amino]-9,10-dioxoanthryl}phenylacetamide. [Method 4]

Prepared from anthraquinone-spacer compound (90) and N-tertiarybutoxycarbonyl-glycine N-hydroxysuccinimide ester (2.2 eq). FABMS(+) m/z: 757 (2%), 735 (100%)(RNH₃)⁺. M, 734. C₄₀H₄₂N₆O₈ requires C, 65.4; H, 5.8; N, 11.4%. Found C, 64.9; H, 6.0; N, 10.9%.

### EXAMPLE (92)

### 2-amino-N-{4-[(5-{[4-(-2-aminoacetylamino)phenyl]amino}]-9,10-dioxoanthryl}amino]phenyl}acetamide trifluoroacetate salt. [Method 6]

Prepared by deprotection of example (91). FABMS(+) m/z: 536 (100%). M, 762

### EXAMPLE (93)

### (2S)-2-[(tert-butoxy)carbonylamino]-N-{4-[(9,10-dioxoanthryl)amino]-cyclohexyl}-3-(4-chlorophenyl)propanamide. [Method 4]

Prepared from anthraquinone-spacer compound (24) and N-tertiarybutoxycarbonyl-L-(4-chloro)-phenylalanine N-hydroxysuccinimide ester. . Mp 190° C. CIMS (+) m/z: 624 (8%)(M+Na)⁺, 602 (95%)(MH)⁺. 546 (35%), 279 (21%), 225 (100%). M, 602.

### EXAMPLE (94)

### (2S)-2-amino-N-{4-[(9,10-dioxoanthryl)amino]cyclohexyl}-3-(4-chlorophenyl)propanamide trifluoroacetate salt. [Method 6]

Prepared by deprotection of example (93). ESMS(+) m/z: 534 (1%), 502 (100%)(RNH₃)⁺, 281 (15%), 132 (55%). ESMS(−)(Cone20V) m/z: 113 (100%), 69 (35%). M, 615.

### EXAMPLE (95)

### 1-azaperhydroepin-4-yl anthracene-9,10-dione trifluoroacetate salt. [Method 1]

Prepared by the reaction of homopiperazine and 1-chloroanthraquinone in DMSO. The solution was cooled and di-tertiarybutyl-dicarbonate (5 eq) in methanol (100 cm³) was added dropwise and the reaction mixture was allowed to reach room temperature. The crude N-^{t}Boc protected compound was extracted into chloroform and applied to a silica gel chromatography column, using toluene:ethyl acetate (4:1) as the eluting solvent, to give the N-^{t}Boc protected compound. The N-^{t}Boc protected compound was deprotected using trifluoroacetic acid [Method 6] to give the title compound. ESMS(+)(Cone20V) m/z: 307 (100%)(RNH₃)⁺. ESMS(−)(Cone20V) mm/z: 113 (100%). M, 420.

### EXAMPLE (96)

### 1-[4-N-tertiarybutoxycarbonylglycyl-L-phenylalanylamino)cyclohexylamino]anthracene-9,10-dione. [Method 4]

Prepared by the reaction of N-^{t}-Boc-glycine-N-hydroxysuccinimide ester (3.3 mmol) and (2S)-2-amino-N-{4-[(9,10-dioxoanthryl)amino]cyclohexyl}-3-phenylpropanamide trifluoroacetate (26) (3 mmol) and triethylamine (2 cm³) in THF (70 cm³). CIMS(+) m/z: 625 (100%)(MH)⁺. M, 624.

### EXAMPLE (97)

### 1-[4-(glycyl-L-phenylalanylamino)cyclohexylamino]anthracene-9,10-dione trifluoroacetate salt. [Method 6]

Prepared by deprotection of example (96). FABMS(+) m/z: 525 (100%)(RNH₃)⁺. M, 638.

### EXAMPLE (98)

### 1-[4-(N-fluorenylmethoxycarbonyl-L-phenylalanyl-L-seryl(ψ^{Me,Me}pro)amino)cyclohexylamino]anthracene-9,10-dione. [Method 4]

Prepared from anthraquinone-spacer compound (24) and N-fluorenylmethoxycarbonyl-L-phenylalanyl-L-seryl(ψ^{Me,Me}pro) O-pentafluorophenolate ester. FABMS(+) m/z: 839 (6%)(M+Na)⁺, 817 (15%)(MH)⁺, 391 (75%), 284 (80%), 225 (100%). M, 816.

### EXAMPLE 99

### (2S)-2-((2S)-2-amino-3-phenylpropanoylamino)-N-{4-[(9,10-dioxoanthryl)amino]cyclohexyl}-3-hydroxypropanamide trifluoroacetate salt

The Fmoc protected compound (98) was dissolved in 20% (v/v) piperidine in DMF (20 cm³) and stirred at room temperature for 5 min. The solution was partitioned between chloroform and water (1:1, 100 cm³), washed with water (3×50 cm³), dried (Na₂SO₄), filtered and evaporated to a low volume before application to a silica gel chromatography column [chloroform:methanol (19:1)] eluting with chloroform:methanol, (increasing gradient,19:1→5:1). The fractions containing the product were combined, evaporated to dryness and dissolved in trifluoroacetic acid. After 20 minutes the solvent was evaporated and dissolved in a minimum volume of ethanol (3 cm³). Addition of ether (100 cm³) gave a precipitate of (99). ESMS(+)(Cone20V) m/z: 576 (2%), 554 (100%)(RNH₃)⁺. ESMS(−)(Cone20V) m/z: 113 (100%). M, 667.

### EXAMPLE (100)

### (2S)-2-amino-N-{4-[(4,8-dihydroxy-9,10-dioxoanthryl)amino]-cyclohexyl}-3-phenylpropanamide trifluoroacetate salt. [Method 6]

Prepared by deprotection of example (84). ESMS(+)(Cone20V) m/z: 500 (100%)(RNH₃)⁺. ESMS(−)(Cone20V) m/z: 113 (100%). M, 613.

### EXAMPLE (101)

### N-{(1S)-2-[4-(9,10-dioxoanthryl)azaperhydroepinyl]-2-oxo-1-propylethyl}(tert-butoxy)carboxamide. [Method 4]

Prepared by the reaction of anthraquinone-spacer compound (95a) and N-tertiarybutoxycarbonyl-L-norvaline N-hydroxysuccinimide ester in THF and triethylamine (1 eq). CIMS(+) m/z: 506 (100%)(MH)⁺. M, 505.

### EXAMPLE (102)

### 1-[1-((2S)-2-aminopentanoyl)azaperhydropin-4-yl]anthracene-9,10-dione trifluoroacetate salt. [Method 6]

Prepared by deprotection of example (101). ESMS(+)(Cone20V) m/z: 406 (100%)(RNH₃)⁺. ESMS(−)(Cone20V) m/z: 113 (100%). M, 519.

Biological Assays

In vitro Activity Against MAC 15A Adenocarcinoma.

MAC 15A cells were grown in RPMI 1640 medium supplemented with 10% foetal calf serum containing a 1% antibiotic mixture under standard tissue culture conditions and were maintained at 37° C. in a humidified atmosphere of 5% CO₂ in air. Cells were harvested from a stock culture in exponential growth phase and plated in 96-well flat-bottomed plates to achieve a final density of 2×10³ cells per well. After 2 hours incubation medium was replaced with either fresh medium containing 0.5% DMSO (control) or medium containing test compound dissolved in DMSO at a concentrations from 10 mM to 1 nM. Chemosensitivity was assessed using MTT assay by the method of Plumb et al Cancer Research 49 (1989) 4435-4440.

Following 96 hours continuous exposure to drug at 37° C. cells were incubated with fresh drug-free medium immediately prior to addition of MTT solution (5 mg/ml). Medium and MTT were removed after 4 hours and 150 μl of DMSO was added. For each plate the absorbance of the resulting solution was measured at the analytical wavelength 550 nm for formnazan product, using a Labsystem Mutiskan. IC50 values were obtained from growth curves of drug concentration against % survival and are expressed in μm. Results are shown in Table 2 below.

In vitro Topoisomerase Assays.

To determine the effect of the newly synthesised compounds on the catalytic activity of topo I and II (α and β), specific tests measuring relaxation, decatenation and enzyme-mediated cleavage of DNA were employed using purified human topos.

It should be noted that the compounds of this study were assayed against each of the purified α and β isoforms of human topo II. In contrast, the majority of published studies on topo II from human cell lines have concentrated on the α-isoform or a mixture of isoforms.

DNA Topoisomerase I Relaxation Assays

Topo I Relaxation Assay Protocol.

10×Topo I Relaxation Buffer;

100 mM Tris-Hcl (pH 7.5); 500 mM KCl; 1 mM EDTA; 50 mM MgCl₂;150 mg/ml BSA

Loading Buffer (Reaction Stop)

5% SDS; 0.25 mg/ml Bromophenol Blue; 25% Glycerol

10×TBE Electrophoresis Buffer (500 ml)

Tris Base 54.5 g; Boric Acid 27.8 g; 0.5M EDTA 20 ml; DNA 4 μl (400 ng); Buffer (10×) 2 μl; Compound1, 10, 25 and 50 μM; Topo I 0.2 μl (2 units); Distilled H₂O to 20 μl total volume.

To eppendorf micro-tubes (0.5 ml) the above solutions were added in the following order: Distilled H₂O, DNA, buffer, compound, and mixed by gently tapping the side of the tube being careful not to disperse the reaction contents. The enzyme was pipetted directly onto the side of the tube and the reactions initiated simultaneously by brief centrifugation. The reaction mixture was incubated for 30 mins at 37° C. following which the reactions were terminated by the addition of 4 μl of the loading buffer. The samples were loaded into the wells of a pre-prepared 0.8% agarose gel prepared and immersed in 1×TBE buffer, and the electrophoresis separation of DNA fragments performed. Electrophoresis was carried out until the blue loading buffer had migrated to around ¾ the length of the gel, typically around 16 hrs at 20 volts, or 3-4 hrs at 60 volts. Each gel was then stained for one hour in 50 μg/ml ethidium bromide in 1×TBE buffer, destained for one hour in H₂O, and photographed.

DNA Binding Assays.

The propensity of selected compounds to bind to DNA in the absence of topos was measured in order to identify compounds that would bind weakly, or not at all. Such compounds were thought less likely to exhibit non-specific toxicity or cause chromosomal damage. A topoisomerase I/DNA unwinding assay was used determine binding constants of the compound to DNA by displacement of either ethidium bromide (an intercalator) or Hoechst dye 33258 (a groove binder) which form a DNA-bound fluorescent complex measured by fluorescence spectroscopy.

DNA Binding Assay Protocol

In order to detect the strength and mode of anthraquinone compound binding to DNA the displacement of known DNA binders was detected by measuring the fluorescence of a DNA/fluorescent compound complex. The addition of known concentrations of ethidium bromide, an interchelator, and Hoescht Dye, a groove binder, cause a fluorescent DNA/binder complex that can be detected and the fluorescence quantified. The addition of anthraquinone compounds displaces the interchelators or groove binders, depending on the compound mode of DNA binding, and therefore reduces the fluorescence accordingly. The preferred binding action of compounds can be quantified by determining the reduction in fluorescence resulting from a given concentration of compound. The quantification of a compounds ability to bind to DNA is expressed, as the concentration required to displace 50% of the ethidium bromide or Hocscht Dye, thus reducing the fluorescence by 50%. Therefore, the values produced are QE₅₀ for ethidium, bromide, or QH₅₀ for Hoescht Dye.

Compound: 300, 60, 10 and 10/6 gM (1.66 μM) solution concentrations were prepared for use in the assay to produce a range of compound. Concentrations. 100, 200 and 300 μl of each dilution were used in the assay reaction. This provides a range of concentrations: −30, 20, 10, 6, 4, 2, 1, 0.67, 0.33, 0.17, 0.11, 0.06, 0.00 μM in the assay environment. Intermediate concentrations can be utilised to enhance the accuracy of the displacement concentration determination.

Buffer: 100 mM Tris, NaCI 500 mM. DNA: Calf thymus sodium salt. Stock solution of 200 μM was prepared in 10× assay buffer. Dilution of DNA therefore provided the correct concentration of assay buffer in the assay cell. 300 μg DNA was used in 3 ml reaction mixture therefore 20 μM DNA concentration. Hoescht Dye 2 μM final concentration, therefore 100 μl of a 60 μM stock solution was added to a 3 ml assay. Ethidium Bromide 2 μM final concentration therefore 100 μl of a 60 μM stock solution was added to a 3 ml assay. Distilled H₂O Water was added to produce 3 ml reaction volume. Order of addition: 1. DNA, 2. Water, 3. Drug, 4. Dye

The assay was completed with both Hoescht Dye and Ethidium. Bromide for each compound. The ethidium bromide is a DNA inter-chelator, Hoescht Dye is a minor groove binder. 100, 200 and 30 gμ of the lowest compound dilution was assayed. This procedure was repeated with all further dilution's. This provided a curve of fluorescence intensity decrease with increasing compound concentration. QE₅₀ and QH₅₀ values were determined by extrapolating the concentration of compound at the point where the fluorescence intensity was reduced by 50%. Controls involving compound only and ethidium. bromide or Hoechst Dye without compound were carried out for each experiment. Two readings for each concentration of compound were performed per experiment, each experiment was repeated at least three times. Fluorometer: Perkin Elmer Luminescence Spectrometer LS 50B.

Spectrometer settings for Ethidium Bromide:

| FROM | TO | EXCITATION |
|---|---|---|
| 570 nm | 630 nm | 546 |
| SCAN SPEED | EX SLIT | EM SLIT |
| 200 | 10 | 15 |
| Settings for Hoechst Dye: | | |
| FROM | TO | EXCITATION |
| 440 nm | 490 nM | 353 |
| SCAN SPEED | EX SLIT | EM SLIT |
| 200 | 15 | 2.5 |

In vitro Cytotoxicity of Spacer Limked Anthraquinone Peptide Conjugates on MAC15A Colon Adenocarcinoma

| EX- | NU:UB | | PEPTIDE | |
|---|---|---|---|---|
| AMPLE | CODE | SPACER TYPE | MOTIF | IC₅₀ μM |
| 11 | 158 | -4-piperidinoxy- | Ala-TFA | 14 |
| 13 | 171 | -L-prolinol- | Ala-TFA | 22 |
| 26 | 150 | Trans-1,4- | Phe-TFA | 3.5 |
| | | diaminocyclohexane | | |
| 53 | 133 | Prop-Pip-Prop- | D-Ala-TFA | 4.9 |
| 57 | 134 | Prop-Pip-Prop- | L-Pro-TFA | 4.6 |
| 61 | 137 | Prop-Pip-Prop- | D-Phe-TFA | 3.8 |
| 30 | 146 | Trans-1,4-di- | Gly-TFA | 6.0 |
| | | aminocyclohexane | | |
| 32 | 147 | Trans-1,4-di- | Ala-TFA | 4.4 |
| | | aminocyclohexane | | |
| 36 | 148 | Trans-1,4-di- | Val-TFA | 4.5 |
| | | aminocyclohexane | | |
| 28 | 149 | Trans-1,4-di- | D-Phe-TFA | 8.0 |
| | | aminocyclohexane | | |
| 15 | 176 | piperazine | β-Ala-TFA | 20 |
| 17 | 177 | piperazine | Ala-TFA | 25 |
| 34 | 200 | Trans-1,4-di- | D-Ala-TFA | 3.9 |
| | | aminocyclohexane | | |
| 40 | 201 | Trans-1,4-di- | L-Pro-TFA | 4.2 |
| | | aminocyclohexane | | |
| 68 | 202 | Trans-1,2-di- | β-Ala-TFA | 6.2 |
| | | aminocyclohexane | | |
| 66 | 203 | Trans-1,2-di- | Ala-TFA | 6.7 |
| | | aminocyclohexane | | |
| 38 | 206 | Trans-1,4-di- | D-Val-TFA | 4.2 |
| | | aminocyclohexane | | |
| 70 | 218 | 1,4-phenylenediamine | Gly-TFA | 2.2 |
| 46 | 220 | Trans-1,4-di- | 2-Cl-D-Phe-TFA | 4.1 |
| | | aminocyclohexane | | |
| 94 | 221 | Trans-1,4-di- | 4-Cl-L-Phe-TFA | 1.0 |
| | | aminocyclohexane | | |
| 44 | 222 | Trans-1,4-di- | 2-Cl-L-Phe-TFA | 3.0 |
| | | aminocyclohexane | | |
| 23 | 231 | 1,5-bis-piperazine | Ala-TFA | 15 |
| 79 | 237 | piperazine | Lys-bis-TFA | 28 |

In vitro Cytotoxicity of Spacer-Linked Anthraquinone-Peptide Conjugates Against HL-60 Human Leukemia

| | NU:UB | | PEPTIDE | |
|---|---|---|---|---|
| EXAMPLE | CODE | SPACER TYPE | MOTIF | IC₅₀ μM |
| 17 | 177 | PIPERAZINE | Ala-TFA | 10.7 |

DNA Binding of Spacer-Linked Anthraquinone-Peptide Conjugates: Fluorescence Quenching Q₅₀ Values for Ethidium (E) and Hoechst Dye 33258 (H) Displacement of Bound Complexes with pBR322 DNA.

| | NU:UB | | PEPTIDE | QE₅₀ | QH₅₀ |
|---|---|---|---|---|---|
| EXAMPLE | CODE | SPACER TYPE | MOTIF | μM | μM |
| Mitoxantrone | Control | Hydroxyethylamino- | — | 0.35 | 1.0 |
| | | ethylamino | | | |
| NU:UB 31 | Control | -1,3-propyl | Pro-TFA | 1.3 | 1.4 |
| 15 | 176 | -PIPERAZINYL- | β-Ala- | 11.4 | 1.6 |
| | | | TFA | | |
| 26 | 150 | Trans-1,4- | Phe-TFA | 6.5 | 2.3 |
| | | diaminocyclohexane | | | |
| 4b | 180 | PIPERAZINYL | H-TFA | 10.5 | 1.7 |

Inhibition of Topoisomerase I Mediated Relaxation of pBR322DNA by Spacer-Linked Anthraquinone-Peptide Conjugates

| | NU:UB | |
|---|---|---|
| EXAMPLE | CODE | DNA-TOPOISOMERASE ACTIVITY |
| 49 | 131 | PARTIAL inhibition at 25 μM; COMPLETE |
| | | inhibition at 50 μM |
| 57 | 134 | PARTIAL inhibition at 25 μM; COMPLETE |
| | | inhibition at 50 μM |
| 55 | 135 | PARTIAL inhibition at 25 μM; COMPLETE |
| | | inhibition at 50 μM |
| 63 | 138 | PARTIAL inhibition at 25 μM; COMPLETE |
| | | inhibition at 50 μM |
| 28 | 149 | PARTIAL inhibition at 50 μM |
| 26 | 150 | NO inhibition between 25-100 μM |
| 11 | 158 | NO inhibition between 25-100 μM |
| 16 | 171 | NO inhibition between 25-100 μM |
| 6 | 176 | PARTIAL inhibition at 100 μM |
| 17 | 177 | COMPLETE inhibition at 25 μM |
| 21 | 179 | COMPLETE inhibition at 10 μM |
| 66 | 203 | COMPLETE inhibition at 10 μM; concentrations |
| | | above this value antagonised inhibitory activity |

Simulation of Topoisomerase I Mediated Cleavage of pBR322DNA by Spacer-Linked Anthraquinone-Peptide Conjugates

| EXAMPLE | NU:UB CODE | DNA-TOPOISOMERASE ACTIVITY |
|---|---|---|
| 28 | 149 | 20% nicked plasmid DNA at 25 μM |
| 26 | 150 | 45% nicked plasmid DNA at 25 μM |
| 17 | 177 | 10% nicked plasmid DNA at 25 μM |
| 21 | 179 | NO CLEAVAGE between 10-100 μM |

Immunoband Depletion of Topoisomerase I

Reductions in the intensity of the (100 kDa) topo I band were observed for NU:UB 150 in comparison with camptothecin. The extent of immunoband depletion by NU:UB 150 above 200 μM was comparable to the effect of camptothecin at 50 μM.. The data provides good evidence for ternary complex (drug-DNA-enzyme) formation in viable whole cells.

Protocol

The method was adapted from Boege and Andersen [Selected novel flavones inhibit the DNA binding or the DNA religation step of cukaryotic topoisomerase I, F. Boege, T. Straub, A. Kehr, C. Boesenberg, K. Christiansen, A. Andersen, F. Jakob, J, Kohrle, J. Biol. Chem., 271, 2262, (1996)]; briefly, 10⁶ HL-60 cells were cultivated for 1 h with and without drugs. Reactions were terminated by sedimentation of the cells (1000×g, 5 min, 4° C.) and subsequent lysis in PBS/NP40, RIPA buffer containing pepstatin and leupeptin. Samples were subjected to SDS-polyacrylamide (8%) gel electrophoresis and proteins that had entered the gel were electrophoretically transferred to nitro-cellulose sheets by the semi-dry method. Immunstaining of immobilised proteins was carried out using a polyclonal Ab of human topo I [topogen], and subsequently anti-human Ig biotinylated whole Ab (sheep)[Amersham], streptavidin horse-radish peroxidase and the ECL system [Amersham].

1. Use of compound of Formula I: 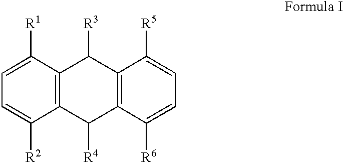
wherein
at least one of R¹, R², R⁵ and R⁶ is a group —AB and the others are independently selected from hydrogen, hydroxy, alkoxy or acyloxy, a group —AB a group -amino-(R⁷)ₙX—Y wherein R⁷ is a divalent organic radical and n is 0 or 1;
R³ and R⁴ are independently oxo, hydroxy or hydrogen;
the or each A is independently a spacer group of formula -amino-(R⁷)ₙ—X— which is bonded to the anthracene ring via the amino group nitrogen and to B via —X—
X is independently selected from O, NH and C(O);
B is an independently selected amino acid residue or a peptide group or isostere thereof and
Y is hydrogen or a capping group,
or a physiologically acceptable derivative of such compound for the manufacture of a medicament for the treatment of cancers or microbial infections having cells exhibiting topoisomerase I activity
characterised in that the group -amino-(R⁷)ₙ—X— incorporates an optionally substituted heterocyclic ring directly attached to the anthroquinone ring through an amino nitrogen in the heterocyclic ring, or an optionally substituted heterocyclic or carbocyclic ring that is spaced from the anthraquinone ring by no more than an amino nitrogen and up to four carbon atoms.

2. Use as claimed in claim 1 characterised in that the heterocyclic or carbocylic ring is a fully or partially saturated ring.

3. Use as claimed in claim 1 or claim 2 characterised in that the medicament is for treatment of human cancers or microbial infections wherein the cancer or microbe topoisomerase I activity is greater than that of non-cancerous or non-microbially infected human cells

4. Use as claimed in any one of claims 1 to 3 characterised in that R¹ and R² are independently selected from hydrogen and hydroxy.

5. Use as claimed in any one of claims 1 to 4 characterised in that the compound is of Formula II

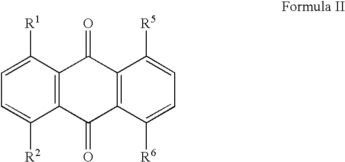

6. Use as claimed in any one of the preceding claims characterised in that only one of R⁵ and R⁶ is a group —A—B and the other is hydrogen, hydroxy, alkoxy, acyloxy.

7. Use as claimed in any one of the preceding claims characterised in that when an optionally substituted heterocyclic ring is present as the -amino- portion of -amino-R⁷—X—, this is of formula is —N<R¹¹—, where R¹¹ consists of a moiety with which the —N< makes up a heterocylic ring system, preferably a single heterocyclic ring, containing the nitrogen atom of the aforesaid —N< moiety and up to 6, but preferably only 3, 4 or 5 other members selected from nitrogen, oxygen, sulphur and carbon.

8. Use as claimed in any one of the preceding claims characterised in that the -amino- portion of -amino-R⁷—X— amino group is a ring selected from NC₄, NC₅, N₂C₃ and N₂C₄ rings.

9. Use as claimed in claim 8 characterised in that the ring is selected from pyrrole, 2H-pyrrole, pyrrolidine, pyrroline, imidazole, imidazidine, imidazoline, pyrazole, pyrazolidine, pyrazoline, pyridine, pyrazine, piperidine, and piperazine and. —R⁷— may be bonded to any of the atoms of the moiety completing the ring.

10. Use as claimed in any one of claims 1 to 6 charactersied in that the or each A is independently a spacer group having the formula —NH—R⁷—NH— or —N<R¹¹—, where R¹¹ includes a further amino nitrogen, which is bonded to the anthracene nucleus via the leading —NH— or —N< moiety and to B via the trailing —NH— moiety or further amino nitrogen in each case.

11. Use as claimed in any one of the preceding claims characterised in that one of R⁵ and R⁶ is hydrogen or hydroxy.

12. Use as claimed in any one of the preceding claims charactersied in that the group -amino-(R⁷)ₙ—X— is selected from:
(iii) those where -amino- comprises a heterocylic ring, which may be optionally substituted, including one or more nitrogen atoms attached to one or more carbon atoms such as to form the amino group and (iv) those where n is 1 and —R⁷— comprises a carbocylic or heterocyclic ring attached to the amino group, preferably the amino nitrogen, or spaced therefrom by no more than one carbon atom, preferably being directly attached to the-amino-group and preferably to its amino nitrogen.

13. Use of a compound as claimed in any one of the preceding claims wherein the compound is of formula III 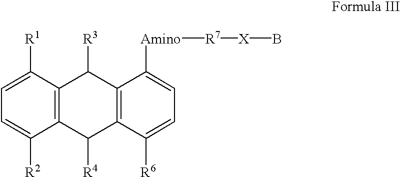
characterised in that amino is a group selected from NC₄, NC₅, N₂C₃ and N₂C₄ heterocyclic rings, ie. pyrrole, 2H-pyrrole, pyrrolidine, pyrroline, imidazole, imidazidine, imidazoline, pyrazole, pyrazolidine, pyrazoline, pyridine, pyrazine, piperidine, and piperazine,m. —R⁷— is bonded to any of the atoms of the moiety completing the ring and amino is bonded to the anthraquinone ring directly through an amino nitrogen.

14. A compound of formula IV 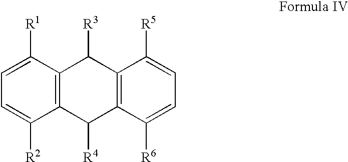
wherein
at least one of R¹, R², R⁵ and R⁶ is a group —AB and the others are independently selected from hydrogen, hydroxy, alkoxy or acyloxy, a group —AB a group -amino-(R⁷)ₙX—Y wherein R⁷ is a divalent organic radical and n is 0 or 1;
R³ and R⁴ are independently oxo, hydroxy or hydrogen;
the or each A is independently a spacer group of formula -amino-(R⁷)ₙ—X— which is bonded to the anthracene ring via the amino group nitrogen and to B via —X—
X is independently selected from O, NH and C(O);
B is an amino acid residue or a peptide group or isostere thereof and
Y is hydrogen or a capping group,
and the group -amino-(R⁷)ₙ—X— incorporates one or more optionally substituted carbocyclic, or heterocylic rings and is selected from
(iii) those groups where -amino- comprises a heterocylic ring, which may be optionally substituted, including one or more nitrogen atoms attached to one or more carbon atoms such as to form the amino group and
(iv) those groups where n is 1 and —R⁷— comprises a carbocylic or heterocyclic ring attached to the amino group, preferably the amino nitrogen, or spaced therefrom by no more than four carbon atoms, or which is directly attached to the amino-group and preferably to its amino nitrogen
or a physiologically acceptable derivative of such compound.

15. A compound as claimed in claim 14 characterised in that —X— is —NH—.

16. A process for preparing a compound of formula IV comprising:
(B) reacting a compound of formula V 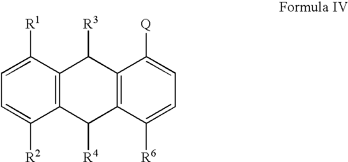
where R¹ to R⁴ and R⁶ are independently selected from those groups as defined in claim 1 and a group Q, and Q is a reactive group such as —Cl, —Br or —OH, with an amino acid or diamine, e.g. an αω-diaminoalkane, to form a compound having the formula V: 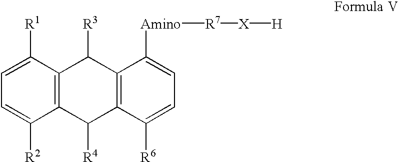
wherein X is —NH— or —C(O)—O— or —O—
and (B) reacting the compound of Formula V with an amino acid or peptide or isostere thereof to give a compound of Formula I.

17. A compound as claimed or described in any one of claims 1 to 15 for use in therapy.

18. A pharmaceutical preparation comprising a pharmaceutically acceptable carrier and/or excipient and a compound for use as described in any one of claims 1 to 13 or a compound as claimed in claim 14 or 15.

19. A method of treating a human or animal body in need of therapy for a disorder selected from the group consisting of cancer and microbial infection comprising administering to said human or animal body an effective therapeutic dose of a compound for the use of any one of claims 1 to 13 or as claimed in claim 14 or 15.

20. A novel intermediate as described in claim 16, forula V.
